SEQUENCE LISTING (35 sequences)

SEQ ID NO: 1 - protein, 411 aa, Spiroplasma citri
MRKLLSIFAATTLVTTSAASAVACSGAPQGNLIPIFMYNGNQKFSHAPTVTRKSINGIDDVTQSGKDENGAPYEYSLQGGRMGLINGLINNAINPILNGINLTKDNSATTGKGAKWTDEQIAAGLEGQKEQLVQTAKTDAQDPFNSSKKINQKAIWKDLFNNYSTSFDSSYSQVAFLANENKAILNKTNDNLVTMTGNAEKTNNKNWVKEHTWPDGKKSPYTPSSLKVLSPIASILEWFNDPKNSYNQGYNQIDQNRGYQSARYLAIAIPNVTIRFEFQGEHNCFTFTVTIDKLVAYANYLVYENPNSTKDNPSYGHQWFFLSYGFYDFESLKDDDYHHYNFNAIPDDVKIDKNIKVALGFFKKNDDKGILTADEDKEVGKRGQFPTAETDYTFPALKWKINVNSITDQYK

SEQ ID NO: 2 - protein, 566 aa, Spiroplasma citri
MKKILSILGAMSILSSTGTSIYACTKKDFERFTDPAIVDEVKRWIIAKINSENESNVVKYSFNDIFTKAALKEMTTRLLDTNISKFFYASEEATRARYTGVTIDVNQPDSLLIKQFINFVEQVALDNLSTKYSTGISNSTPLETTIAGQGYAPDYQSEGWYVGGSQSYFWKGTGKPNYMQSRKGQGEANFEIKSKSAGANYTDFNAMSEDDKKAALKTRFKDYYTHVEIPAVIDKIITATYLHQNEIKRYGSGNNSSIYLNRNSALFNALQSWDTISGARWKSYIKMVWELKLDKEELDKLFADKGPLANVEKLNSDLTNNQDILMKILKEMFNAKKDNIFNNMIKDGIDPIFGISGFKGFVGIDKNKNPNDIFTTLNNADSYKQKVIDTATPGIIKSGEGTDPSSYQFLDTNKRYGSFVLTLPIYAVDLMKNMNINYKNDNKNNKELSLTWYGSGGTPTDLDQAWLAQQGGIKRSLSWLYNKKGYLGTYDENGQPLYNDNGTPVDITKNIKGQILKWIEYTFAKQQNLQTAAKTRLYSLVFANNPENVYSQTLYDAIGSYIIKED

SEQ ID NO: 3 - protein, 730 aa, Spiroplasma citri
MRKLLNILAAATLAATPALTASCKTKAKSAEDKYKDSSVENLPNGPLKSKILQTTLFTKATIANRHENLNTYTPSMLQMLMRLPDSYKDKDGNIVDIDYYRGKYLNKNNGMPLTTLSSNYDYMNLLDNELYNTEKQTKKKISDYSDKDPLPSIPNLTKNSNMLNYWYDGGPLSNYSIVKEILPTKCDDKRINQNIVDACNKAIFEMPRTTYFYNFNMDYSPMATKDIKFDTDTNQNFIINNQKFAAGGPFKTAQKSEEQDKLSIILQLISMADMFTDRSQSKTYINQLNKFLALSGDSDGTFMGSILGAIYYQIFASPKLPNDPTKENANYTFAKLGVTKALQLLRKDSTERQAIKQKIDVFFDAQSQVFSDLLAVRPMQPGLDLNKPEGQYKNRDAMWNGKTPNLRLVDLLFKKDASTKSLAELFTDFGEYLDDLYTKADVECQEEANNSISSFLKLAANVVTPGFKVVLQSMSKMMLSKSEGGLGTLSVNDINRFVVALSKGILQSANALTEVSKLPWSEATDQEQNQTKITQLLTGSDDPSTPTKDSFMDLAFTWFNDSTQPVRALLNKLYFDPDSETRKDLLAINNALYEYSNNLLLGANWNISNGQLEENKLSYDIEYKGTGDADVVANLDLHQNWYIPKSEIKTYQDLNAAYLNALGNRDLDWFMKYDGLGNNYQKVHYKYKVTWMNINPGDDSHQYWVISNIQWFAKDISGQWKRYYDAIEND

SEQ ID NO: 4 - protein, 291 aa, Spiroplasma citri
MKWLLTLFSVFVLGFGSSLGVVSCTVRAKHEPDDNDELDHNQDLEILNQIKKEAKQTLSTWWQTKTMIDIIKDYQEQISSFKELVTQVKTKNDGSLTLTSIAISKYCFLNQLLIGFKAEFNNLNQHLQNRYSNYYVDTMPLFLGENDISFNLYNINFDKIAKLLADTAQAVLGITVQVNIAYEVRFKGLPTEDNIQVSITTTNDSEVLNNIQDKVENYFVNFLDTIFKAKNYRIISKQFNIKTDIVWPIISKELNDRNISFQHIIFNFLYYGQQIYLIFIPLNIWTLTRLF

SEQ ID NO: 5 - protein, 308 aa, Spiroplasma citri
MAKIGGKSSIALPTLFEIGGNSPTEKAIGDVAVLQIKKILEADSQYIDATEMMMMSAKQISMEDGFEQGQYVFPERMVWGNDYDSSAGAEQQSVGVRRATVMMDQMLTFKYDVPSFDTVRFMESPVEVRTNTIGEWMRTITRNWYSNMNAIYLQGVIDSCIATGQYIILPIPTDADSAQQTFYKINDIAINLVQKINALMFGTKKEDLMVHVAMPAFAQFTKAYTKILDQIAADTLATGQLWRKMIVGVDVFESWYLGRQFNKGKETGINKDLDFNLNFSQTVGAWGFIGHKEDCAMPQGWKSIQQVN

SEQ ID NO: 6 - protein, 373 aa, Spiroplasma citri
MKRFFTLLAVLNVATGSTVMVASFTIAQGAHLNPVDSLLLIGKDIDTSKAIDDAKSNLQFTNYYILGDSLSDSHGIEKLVKNSFKLDIKIGTNDPNNLENYQNGSLSNGNTAAVLLNAKLGFDKIRPGIPNDYAGDFGRNYAIGGATAVDVVGTAGMLLNRVTIEKQAQALVSQHKLRSTDLVLFEIGGNDLFQIIDTTDPQTELELMHQSVERIKIALFTLLNNGIRKILFSDAPNVSAIPRYNNQNTDDTLKKRANNISTEFHARVAKMIELANTYYKNAIRNWGLYDNLSVLMTEFKARHPKGNITVNFNNLNLDFIKIIEEKMLNAQRNPALPANANIDDYFFFDIVHPTREVHQLAMEHYYQTIKEWT

SEQ ID NO: 7 - protein, 463 aa, Spiroplasma citri
MLRQFGGLIGSNSLTFGCMLKLTDKIKTHYYSATGQDLGTKVYSTIDLGQNRINVNGGTDEIVILSQDFQALDKDTPLTNLDNKLDFNPTVDGTLESYVIDLINLQTIGKTNFKITTYSENPFTLNDNITAFQNFQGEWQTMAKFIKPNGNVQAWNSVIKTSALDYDLLEETTFFYPQAVLPPDPFTYKPYTVNILDSNTIKPLKANITAAKQCTSSISFIWQKDWINLFNNKYDNVYYELEVDLKQINTTITTWEQLLNAYSSIQFNNLNNLQVKCAPPNIENFIPNRNINILGGYSTCENNLGWFSNGKLKVKGDKQIDKVNFNGSLIYDLANIKENKKTTLFTGETTGLPSGYYQNRYSFSKNCDFSFLLQITKLSDNDDNKIKELYVQAWHTNDMKLKIRFTKVVFPDFSFYYSDMPVVKLYKYFGNDKNISIDIDNSNILGIRNDSINPISITFIPRS

SEQ ID NO: 8 - protein, 1067 aa, Spiroplasma citri
MSRINPSVMEELRRQAHDTLIDIRENIRDNRHWMVFPFNVYSSYIPDTETGWKPNNAKPSDYGSYTRIFDNMESTIAQWQGSLKIVQKYKGEEINILTIDDFNKQPINSTIWANNETPFAIKLKDENENNYWKLIDGENIYDINKMLEYLKQYKLSYFINNDNKADNFYTKEVKVGKYWDAYMIDDRSSIQFGDAKFEQVFTQQSTERQYTLLILDEDAEKLNINDYYNFYTVFNDRGLNIGGGDINSAPQDRQWLYPSNVINYYSPYDGSFLWQEITFSSINDKISHSGLSVRQFIQTSAPGEGYCFPKITYDKKLNKGIVELDEVLLKDPGVRAMAVKFYGNPILFDMPVIGRPIIKGEFNKKVMNSRDLLMYNMYPAPPDKINTYAAPEVSWYNIQGIQPTMITGYEDWKKDMESRYDFWNQKNSEGIVITDKVTSTATNPTVGTTINEEDNNSEFYWSKSWKITPPNKVKVNSGSVDNIISTQASSYFRRKDEFGNTVGQVQIEYNKMGSCNVWDILNINNFINRQITVLPLNYTHKLVFNPFTIPGGVGKFLNFISFGIPWGWVINQDKKTWPNFQWLNGFMSANVYSFYNDAFWSEKSKGKGYLPFEIFREQGNDKVGAIFGANATSLGFTTLLTDKCKGTVYTNDGKITEQITYSTYNLKQLNEKTNRIYLINEQTKAVEAQTPVSDSEKDNDCEFLQTPDGTNCSYIIDMFSIQALYKGNFEIIFYADNPYTNNEEDYLRLSVWSFRGKTKSVLNNYLRDMTTNYKTSFLLPHDYEIPTFNYPQYVLPPVPYNYQPYTKDIMPAGVVQTLKTKITAPAQCKENFPPVNLFDDTAEHNAYYETEIDLRQIDPTIQSIDKFKSAYKTIEISNLGNLQVECGPPNIENFIPNRNVDILGGLNCNINLGSLANITLQESGRSQTDKVNFDNIRTLYTNNITNEVQTFEPIAYTNGLQGDDYENRYFASDKRVVLDFLLQITKLSNNNDNDKIKETYLQAYYTNDVKLKIRISKIININLLLDSTMHWKYFGNNKNIVLDVDNPNVLGIRNKSENPVKITFIPR

SEQ ID NO: 9 - protein, 98 aa, Candidatus Liberibacter asiaticus
MRHLILIMLLSILTTNIARAQVYHIHSPRIATKSSIHIKCHSCTLNKHHINKTPSSSSAVYTKKEELIDGKKAMITTDNFMGGEPITFIKYLFEEDKK

SEQ ID NO: 10 - protein, 351 aa, Candidatus Liberibacter asiaticus
MGLKKFFLGTVAVATIISYSESFAYVRGKTSMVSNNRARNRSAGKVGNVLPHITKVGGSLEKSLQARYHKLNGNNEFNSLAYDIPVKGNLEVNANAGDVTGVAKLKLAVDDVLSMQFAESDVRALAFTVPSSKLSVEELSLSMKGARLGYYKSWSDEVNPVYSPTTLYNDARGLDKMMSLSYRHSFGLLKAGLSTDLLQKDGLKQVLGIGYMASYAIGKIRSTVTGGYDAGTNNVAIRANISSPVSRAGTLDCGAVWASGDNSYYDKSKYSVFAGYKFDVAKSITISGGGQYFGDINKTGKDGWSAGISAKYMISSGLEAQASVAFNDNFVKKGVAIDKGVDLSVGLKKSF

SEQ ID NO: 11 - protein, 309 aa, Candidatus Liberibacter asiaticus
MYKILAVCLFLTTFSISYARDADSCTPVRFADTGWTDIAATTAMTSVILEEILGYKTNIKLLAVPVTFRSLKNKGIDIFMGYWYPSLEKFIAPYLEEGSIKLVAENLQGAKYMLAVNDVGFALGIKSYQDIAKYKKELGAKIYGIEPGNEGNQRILDMINNNKFSLKGFRLIEASELASFSQIRRDQRNNIPAVFLSWEPHPINSDLNIHYLPGGEEISGFGEASVYTVVRSDYLDKCPNISRLLKNIKFSVALENEMMKLILNNKQDRQFVGRTMLRTHPDLLKNWLIGVTTFDGQDPSRQLERFMNN

SEQ ID NO: 12 - protein, 238 aa, Candidatus Liberibacter asiaticus
MYRYSFIVLCYSSLLFGCHSSISEITGIPQMSPMGSSLDENNRMPFLGIDFKNSDSTKKSYSLWRDSHAALFKDSRALNVGDILTVDIRIDDQAVFDNQTGRSRNNSLHRKLSGGFSLFGQQTPQMNGNLNYDGGGASSGKGSISRAEKLNLLIAAIVTAILENGNLIISGSQEVRVNDEIRSLNVTGIVRPQDVDAHNSVSYDKIAEARISYGGKGRTTELLRPPIGHQLIENLSPL

SEQ ID NO: 13 - protein, 175 aa, Candidatus Liberibacter asiaticus
MILLPIIYYYKKRDMLSQLLFLLFFFLQGFANQSYGDPTLVDREIQQYCTNVIDSVRERDYLSQKKVLEDLQKDIEQRVILLENHKKEYNLWFQKYDSFIMSYNKNILDIYKKMDSDSAALQLEQIDPDISSHILMRLSPRQSSLIMSKMNPKSATMITNVVANMLKFKKLKRSS

SEQ ID NO: 14 - protein, 336 aa, Candidatus Liberibacter asiaticus
MRQFLVIIAMAMLSNIFPFLSIAETNKLPYYTLLDTNTGHVIAENYPDHPWNPASLTKLMTAYVVFSFLKEKKAMLTTPITISKNASEYPPSNSTFKKGSTMTLDNALKLLIVKSANDIAVAIAESLCKTEKKFVQHMNNTSKNLGLSATHFMNAHGVVQHGHYTTARDMAILSWRIKTDFPQYMHYFQIKGLEIKGKKYPNTNWAVGTFLGADGMKTGFTCASGFNIVASAIQGDQSLIAVILGALDRNTRNKVSASLLSMGFYNKTDRKKINYIIKDFYQQNLSNEVPNISEEVCTTQKEIINYNTQMKEAKDKESHFIDFEKITLLKNKITKK

SEQ ID NO: 15 - protein, 162 aa, Candidatus Liberibacter asiaticus
MNFRIAMLISFLASGCVAHALLTKKIESDTDSRHEKATISLSAHDKEGSKHTMNAEFSVPKNDEKYTISSLTKKIESDTDFRREKATISLSAHDKEGSKHTMNAEFSVPKNDEKYTISACASDDKGNKSTLCVECPSPSTPGQYDLNHCAECENTTSKGLCP

SEQ ID NO: 16 - protein, 474 aa, Candidatus Liberibacter asiaticus
MRYLQRTFFTMMSIFLFSSNPSVAKLPPIKEANAAVINISDVEIGKGKKISIGLNKVIILQVPVDVQDVLVSDPTKADVVVHSPRTMYLFGKNVGQANVILIGHDGKQMLNLDILIERDIAHLEMTLRRFIADSNIRVEMVSDTVVLHGMVRTIQDSQRAVELSETFLSQSGRNQYANSSSKKVMNLLNIAGEDQVTLKVTIAEVRRDILKQIGFQHSITGSSSGPSKSFAADFGGKFVSEGGDFSVKGVLDRFSFETVLHALERATAIRTLAEPTLTAISGQSASFTSGGQHLYKTVSSSTGATSVTTHDYGVVLHFTPTVLSPGRIGLRIQTEVSEPVIGVNAGDMPSYRVRKADTTVELPSGGTIVLAGLLKDDIQQLKEGIPLLSKIPILGALFRNSRFNREETEIFIAATPFLVKPVAMRDLSRPDDHYSVEDDAKAFFFNRVNKIYGPKEASEVEGQNYKGAIGFIYK

SEQ ID NO: 17 - protein, 412 aa, Candidatus Liberibacter asiaticus
MYFITIISIVFTLPSHALSIVPNVSLEKVLQNYATIAHAKYEDALMCARTLDSAIETLVTTPNKKNLENARLQWIRARIPYQQSEVYRFGNKIVDTWDKKVNAWPLDEGFIDYVDSSYGKENEENNLYTANIIANSKIIVNEKEIDLSIISPDLLRKLHRANGIDTNITTGYHVIEFLLWGQDLKTNVREPGNRPYTDFDIGNCTGGHCRRRVEYLKVVSKILVSDLEEMMKAWGPDGQATKDLMKDINAGLNSIITGMTSLSYNELAGERMNLGLILHDPEQEIDCFSDNTYASYLNDVIGIISSYTGEYIRMNGEKIHGASIHDLISHNNRNLAQEINDKFSNTMKDFHILKDRAENIESFDQMISENNPEGNKIVRNLINDLITQTESLRKIRIALDLIEPNRVIGNVP

SEQ ID NO: 18 - protein, 131 aa, Candidatus Liberibacter asiaticus
MCRKIIFALTIIAIAFQSMALNCNETLMQADMNQCTGNSFALVKEKLEATYKKVLEKVEKHQRELFEKSQMAWEIYRGSECAFAASGAEEGTAQSMIYANCLQGHAIERNEKLESYLTCPEGDLLCPFINN

SEQ ID NO: 19 - protein, 205 aa, Candidatus Liberibacter asiaticus
MQKLFLAVGVSSLALASFCSAQAADPVRRAHHGGRGVVPTIATNRYVPIRHDFNGPYAGLSALYNGSFGEEAHHNAGGSIFAGYNVEDSCIMYGVEGDVRYTVPVLADNIHSLHGIGGSLRIRGGYEVSDSLLLYATVGPDVAQKYETGKAGEITPIAIGGTAGVGVEVGGLSESLVARLEYRASKYSKVEGFYNTISLGVGMKF

SEQ ID NO: 20 - protein, 195 aa, Candidatus Liberibacter asiaticus
MFKRTIYTYLLLLCGFTEAFSTENTTKYLTLYTDQNQSVMLPIIHSFEERTGVKISPIYTSSIQRPPITQGSPVDVIITKDETSLALNEDLLHKLPAHLIKKNSFVLKNENKKLMRISFDTQVLAYSTKRIKIADLPKSVFDLTNAQWKKRLSIAPNNISFHRLLNTMEQTPNKTVVQDFIKNITANEILTKYKR

SEQ ID NO: 21 - protein, 210 aa, Candidatus Liberibacter asiaticus
MKYRVLLLILFFVFSHAKFANSARFANKVAEFAGMDKITGRVLTFDVEINQSAQFGSLIIKPMVCYSRDDREAQRIDAFVSISEIFTDRIVRSIFSGWMFADSPAMNAIDHSIYDIWLMQCKDPINDSISNSESISKKALSEYSSTDITSQGSEKSSGSSSNKTLEKESSQPLENNLSMDLKGRPIQELGNNLSDSGLNEQDHNDVQISK

SEQ ID NO: 22 - protein, 294 aa, Candidatus Liberibacter asiaticus
MLRYFICLLFSYIPMSASATTQKKVVLSSFSIIGDITQNIAKDLVTVTTLVEAGNDSHSYQVTSADAIKIQNADLILCNGLHLEETYMKYFTNLKKGTKIITVTDGINPIGVSEDTSVDSEPNPHAWMSLTNAMIYIENIRKALTALDPSNAKKYELNAREYSEKIRNSILPLKTRIEKVDPEKRWFVTSEGCLVYLAEDFGFKSLYLWPINSDSERSPSMMRHAINQMRSHKIKFIFSESTNSDQPAKQVAYETNASYGGVLYVDSLSKPDGPAPTYLDLLRFSLTKIVDTLF

SEQ ID NO: 23 - protein, 96 aa, Candidatus Liberibacter asiaticus
MTISKNQAILFFITGMILSSCGDTLSDSKQHNKINNTKNHLDLLFPIDDSHNQKPTEKKPNTSSIKIKNNIIEPQPGPSRWEGGWNGERYVREWER

SEQ ID NO: 24 - protein, 159 aa, Candidatus Liberibacter asiaticus
MKRLKYQIILLSLLSTTMASCGQADPVAPPPPQTLAERGKALLDEATQKAAEKAAEAARKAAEQAAEAAKKAAEKIIHKDKKKPKENQEVNEVPVAANIEPESQETQQQVINKTTTSQTDAEKTPNEKRQGTTDGINNQSNATNDPSSKDKIAENTKED

SEQ ID NO: 25 - protein, 276 aa, Candidatus Liberibacter asiaticus
MKRFSCDCLLKGSVVCVVVLGMSSCFFSSTYKDDKLEYFPESMYGVTASDRIVSGKRVPRGGGRYFLGKPYQIMGRWYVPRQYTAYAAVGMASWYGKAFHGRLTANGEVYGTEYITAAHPTLPLPSYVRVTNMENGISLVVRVNDRGPYHSNRLIDLSNAAAKILRVEERGVSKVHVEYLGMALLNGMDQEYLRSTVMVNSATVLPLGCQYREEIVVIPYLLTRSRTVHLNNCDDDSLQKQREISLRERKKSNLIPLPNGYSPPRKMGKIPIPSRF

SEQ ID NO: 26 - protein, 304 aa, Candidatus Liberibacter asiaticus
MIRKYVLALVFFLVPCTASVAQKVRLVSWNINTLSEQEGVSLWKNSVKRTTSDYTLLRQYAKNLDADIVFLQEMGSYNAVAKVFPKNTWCIFYSTERLINHSKRDSNNDIHTAIAVRKKNVRVLQQSYPLLGAKDSFSRAGNRRAVELLVEINGKKIWVLDIHLKSFCFLDSLENTYSPSCSLLSQQAQWLKDWITQKKESLVPFVIAGDFNRKINYLGNNDDFWKTIDPNDSLIRFPKEKDSRCNANKNLRNKIPIDYFVMDQNAYKFLIQESFSEILYNEDDIKSRGKRLSDHCPISIDYDF

SEQ ID NO: 27 - protein, 116 aa, Candidatus Liberibacter asiaticus
MFWIAKKFFWISVLLIVLSNVYAQPFLEETEKGKKTEITDFMTATSGTVGYASNLCNAKPEICLLWKKIMRNVKRHTLNGAKIVYGFAKSALEKNERESVAIHSKNEYPPPLPSHH

SEQ ID NO: 28 - protein, 185 aa, Candidatus Liberibacter asiaticus
MFLNVLKDFFVPRIRFLIVLMVSSVSAGYANASQPEPTLRNQFSRWSVYVYPDLNKKLCFSLSVPVTVEPLEGVRHGVNFFIISLKKEENSAYVSELVMDYPLDEEEMVSLEVKGKNASGTIFKMKSYNNRAAFEKRSQDTVLIEEMKRGKELVVSAKSKRGTNTRYIYSLIGLSDSLADIRKCN

SEQ ID NO: 29 - protein, 154 aa, Candidatus Liberibacter asiaticus
MRKNLLTSTSSLMFFFLSSGYALSGSSFGCCGEFKKKASSPRIHMRPFTKSSPYNNSVSNTVNNTPRVPDVSEMNSSRGSAPQSHVNVSSPHYKHEYSSSSASSSTHASPPPHFEQKHISRTRIDSSPPPGHIDPHPDHIRNTLALHRKMLEQS

SEQ ID NO: 30 - protein, 208 aa, Candidatus Liberibacter asiaticus
MDKKLNKIIKKKIISCSLAVLLCTSLSSCFFHNNPVNIYDLTESTKYDESVQRHIQLIITEPITEKILNSEDIIVRSSPIEIQYLIGSQWSDKLPRMIQLKLIANFENNGKISTVVKPNQGIYADYQIISAIRSFEINIDRHCAIITMSLKIINAHDNSLVGQKVFHVEEKLEKDNKLHFIQSLNRAFSRISSEIIDWTLSSLPLSDN

SEQ ID NO: 31 - protein, 396 aa, Candidatus Liberibacter asiaticus
MNQKYLICTMMVAMDVFFSFATDQDLVRTIVPYQCVRSLQRALDEAMRGDISLQKKIPDIVKETGVQLRATHMDVFVDNRNIDAVWIYTIISQDLSVVDDLIAKDTKGYFDIAIVYALKKYFSGQLEESSKELSKIKDKDNTRGIVPYLHLLIGRAMMPFSSQQAVHFFDYVRLTSPGTFLEEIALRNLLEITQNEVGERAFGYIRAYVTQFHHSIYKDHFISVLLRFFLHGQLKLPDEDIVFTISFFSLEEQRAIYLKIAQNSVISGKRKIGFLAIKQLKRIIDRLDYKDLATIQLYENILNIPFVDIMSLQRSTCNIPYYSLMEQDRYLKKASEIIMSEIGKSLIDIDFEHIQKDLLLDKKEPRHTNVSMGIESFIKKNRSQIESIDVLLAEAR

SEQ ID NO: 32 - protein, 60 aa, Candidatus Liberibacter asiaticus
MARTQIALALSFFMITHSYYAFSQDEIKKNNPTLEKKPIVLMKHEIQEKKTLAAFTSFAS

SEQ ID NO: 33 - protein, 195 aa, Candidatus Liberibacter asiaticus
MKSKNILIVSTLVICVLSISSCDLGDSIAKKRNTIGNTIKKSINRVIQENNKPRNMTIFKTEVKRDIRRASRLSLEEKSKNADKPTVIENQADNINIEVEVATNLNPNHQASEIDIAIENLPDLKSNHQASEIDIAIENLPDLKSNHQASEIDIAIENLPDHQVDRNHTLSNLRGACYQPSLVSNSSLKLWDVAF

SEQ ID NO: 34 - protein, 68 aa, Candidatus Liberibacter asiaticus
MTIKKVLIASTLLSLCGCGLADEPKKLNPDQLCDAVCRLTLEEQKELQTKVNQRYEEHLTKGAKLSSD

SEQ ID NO: 35 - protein, 85 aa, Candidatus Liberibacter asiaticus
MSKFVVRIMFLLSAISSNPILAANEHSSVSEQKRKETTVGFISRLVNKRPVANKRCPNATKQTPPDHGSKYDTREVLMLFGGLNN

(19) United States
(12) Patent Application Publication
(10) Pub. No.: US 2019/0195873 A1
(43) Pub. Date: Jun. 27, 2019

(54) IDENTIFICATION OF SECRETED PROTEINS AS DETECTION MARKERS FOR CITRUS DISEASE

(71) Applicant: The Regents of the University of California, Oakland, CA (US)

(72) Inventors: Wenbo Ma, Riverside, CA (US); Georgios Vidalakis, Riverside, CA (US)

(21) Appl. No.: 16/158,131

(22) Filed: Oct. 11, 2018

Related U.S. Application Data
(63) Continuation-in-part of application No. 15/069,441, filed on Mar. 14, 2016.
Application No. 15/069,441 is a division of application No. 13/829,270, filed on Mar. 14, 2013, now Pat. No. 9,389,230.
(60) Provisional application No. 61/615,760, filed on Mar. 26, 2012.

(51) Int. Cl. G01N 33/569; G01N 33/92; G01N 33/58; C07K 16/12

(52) U.S. Cl. CPC G01N 33/56911; G01N 33/92; G01N 2021/8466; G01N 33/581; C07K 16/1264; C12Y 111/01007

(57) ABSTRACT

Secreted proteins as detection markers for insect vector and graft transmitted citrus disease are described. Method and kits for detecting the secreted proteins are provided.

### CROSS-REFERENCES TO RELATED APPLICATIONS

This application is a continuation-in-part of U.S. patent application Ser. No. 15/069,441 filed on Mar. 14, 2016, which is a divisional of U.S. patent application Ser. No. 13/829,270, filed on Mar. 14, 2013, which claims benefit of priority to U.S. Provisional Application No. 61/615,760, filed on Mar. 26, 2012, the disclosure of which is hereby incorporated by reference for all purposes.

### SEQUENCE LISTING

The instant application contains a Sequence Listing which has been submitted electronically in ASCII format and is hereby incorporated by reference in its entirety. Said ASCII copy, created on Jan. 8, 2019, is named 081906-1104075-211230US_SL.txt and is 89,266 bytes in size.

### BACKGROUND OF THE INVENTION

Citrus production is one of the most important agricultural economic activities in the United States and around the world. According to the Food and Agricultural Organization (FAO) of the U.N., approximately 47,170 tons of citrus fruits such as oranges, lemons, grapefruits, etc., were produced in 2011, corresponding to US$2.3 billion from sales of fresh fruits and juices around worldwide (FAOSTAT). However, the citrus industry has been experiencing a big threat from phloem-colonizing and insect-transmitted bacterial diseases including Citrus Stubborn Disease (CSD) and Huanglongbing (HLB, also known as citrus greening). Diagnosis of these diseases has been very challenging because of the low titer and uneven distribution of the pathogens in the citrus tree.

The causative bacteria Candidatus Liberibacter (for HLB) and Spiroplasma citri (for CSD) reside exclusively in the phloem of infected trees once they are introduced by the phloem-feeding insect vectors or by grafting. So far, CSD or HLB resistant varieties of citrus have not been found.

The most commonly used pathogen detection methods are PCR based, which requires the presence of bacterial cells or DNA on the tested sample for positive diagnosis. This is been problematic because the low titer and variable distribution of the pathogen within infected trees. Both S. citri and Ca. Liberibacter cells exhibit extremely uneven distributions in infected trees. Very often, the pathogen cells cannot be detected even in symptomatic branches or leaves. Moreover, nucleic acid-based assays require sample preparations, which can be complex, costly and time consuming, especially when numerous samples have to be tested for one tree. So far, the ability to process thousands of samples necessary to track an epidemic using nucleic acid-based methods remains manpower and cost challenging.

Bacteria pathogens secrete numerous proteins to the environment and some of these secreted proteins are essential virulence factors functioning in plant cells. Genes encoding these specialized pathogenesis-related protein secretion apparatus are present in the host plant, but absent from S. citri and Ca. Liberibacter. Bacterial pathogens are injected into plant tissues, i.e., the phloem, by their corresponding insect vectors at the initial stage of infection; therefore, proteins secreted from the general secretion system are readily delivered inside the host cells. This is consistent with the observation that the pathogen cells are often absent from symptomatic tissues. While not being bound by any particular theory, it is believed that the presence of secreted proteins are responsible for the symptom development. Importantly, there are no curative methods once the trees are infected. The success of disease management is largely dependent on early pathogen detection.

### BRIEF SUMMARY OF THE INVENTION

One aspect presented herein is a method of detecting citrus stubborn disease (CSD) in a citrus plant. The method includes detecting the presence or absence of a secreted protein from Spiroplasma citri in a sample from the plant, whereby the presence of the secreted protein indicates that the plant has citrus stubborn disease. In some embodiments, the sample or sap is not subjected to protein separation or other methods of isolating, extracting or purifying proteins in the sample prior to the detecting step.

In some embodiments, the secreted protein for detecting CSD is encoded by CAK98563, or is substantially identical to the protein encoded by CAK98563. The CAK98563 protein (SEQ ID NO:1), also referred to as ScCCPP1, is predicted to be a transmembrane lipoprotein. In some embodiments, the secreted protein is encoded by CAK99824, or is substantially identical to the protein encoded by CAK99824. The CAK98563 protein (SEQ ID NO:2) is predicted to be a transmembrane lipoprotein. In some embodiments, the secreted protein is identical or substantially identical to a protein listed in Table 1.

Another aspect presented herein is a method of detecting citrus greening disease (Huanglongbing or HLB) in a citrus plant. The method includes detecting the presence or absence of a secreted protein from Candidatus Liberibacter asiaticus in a sample from the plant, whereby the presence of the secreted protein indicates that the plant has HLB. In some embodiments, the sample or sap is not subjected to protein separation or other methods of isolating, extracting or purifying proteins in the sample prior to the detecting step.

In some embodiments, the secreted protein for detecting HLB is identical or substantially identical to a protein listed in Table 2.

In some embodiments, the citrus plant is not artificially infected or graft-inoculated with a bacterial pathogen.

In some embodiments, the secreted protein is detected by detecting the specific binding of an antibody to the secreted protein. In some instances, the antibody binds to the secreted protein encoded by CAK98563, or to the secreted protein substantially identical to the protein encoded by CAK98563. In some instances, the antibody binds to the secreted protein encoded by CAK99824, or to the secreted protein substantially identical to the protein encoded by CAK99824. In some instances, the antibody binds to a secreted protein that is identical or substantially identical to a protein listed in Table 1. In other instances, the antibody binds to a secreted protein that is identical or substantially identical to a protein listed in Table 2.

In some embodiments, if a plant is determined to have CSD (Spiroplasma citri) or HLB (Candidatus Liberibacter asiaticus), the infected plant is removed and or destroyed.

Another aspect presented herein is a kit for detecting citrus stubborn disease. The kit contains one or more reagent specific for a secreted protein from Spiroplasma citri. In some instances, the reagent is an antibody. In some embodiments, at least one reagent of the kit is an antibody that specifically binds to the secreted protein encoded by CAK98563, or to the secreted protein substantially identical to the protein encoded by CAK98563. In some embodiments, at least one reagent of the kit is an antibody that specifically binds to the secreted protein encoded by CAK99824, or to the secreted protein substantially identical to the protein encoded by CAK99824. In some embodiments, at least one reagent of the kit is an antibody that specifically binds to a secreted protein that is identical or substantially identical to a protein listed in Table 1.

Another aspect presented herein is a kit for detecting citrus greening disease. The kit contains one or more reagent specific for a secreted protein from Candidatus Liberibacter asiaticus. In some instances, the reagent is an antibody. In some embodiments, at least one reagent of the kit is an antibody that specifically binds to a secreted protein that is identical or substantially identical to a protein listed in Table 2.

In some embodiments, the antibody of the kit for CSD or HLB is labeled. In some instances, the antibody has a detectable label (e.g., fluorescent dye, enzyme, biotin, etc.).

In some embodiments, the antibody of the kit for CSD or HLB is linked to a solid support. Non-limiting examples of a solid support include the surface of an ELISA plate, a glass slide, a coated plate, a bead such as a silica, plastic or derivatized plastic, paramagnetic or non-magnetic metal bead, a polymeric gel or matrix, or a filter, such as a nylon or nitrocellulose.

In some embodiments, the kit for CSD comprises more than one antibody that specifically binds to any one of the proteins (or substantially identical variants thereof) described in Table 1, wherein a first antibody that binds the protein of interest is linked to a solid support and a second antibody that binds the protein of interest is labeled with a detectable moiety.

In some embodiments, the kit for HLB comprises more than one antibody that specifically binds to any one of the proteins (or substantially identical variants thereof) described in Table 2, wherein a first antibody that binds the protein of interest is linked to a solid support and a second antibody that binds the protein of interest is labeled with a detectable moiety.

Also provided are antibodies (optionally isolated) that specifically bind to secreted proteins as described herein. In some embodiments, the antibody specifically binds a secreted protein that is identical or substantially identical to a protein listed in Table 1. In some embodiments, the antibody that specifically binds to the secreted protein (i.e., SEQ ID NO:1) encoded by CAK98563, or to the secreted protein substantially identical to the protein (i.e., SEQ ID NO:1) encoded by CAK98563. In some embodiments, the antibody that specifically binds to the secreted protein encoded by CAK99824 (i.e., SEQ ID NO:2), or to the secreted protein substantially identical to the protein (i.e., SEQ ID NO:2) encoded by CAK99824. In some embodiments, the antibody specifically binds a secreted protein that is identical or substantially identical to a protein listed in Table 2. In some embodiments, the antibody is detectably-labeled. In some embodiments, the antibody linked to a solid support.

Also provided is a method of detecting secreted protein in a citrus plant, the method comprising forming a mixture of a sample from a citrus plant with a first antibody specific for a protein identical or substantially identical to a protein as listed in Table 1 or Table 2 or a protein comprising a fragment of at least 20, 30, 40, 50, 70, or 100 contiguous amino acids thereof; incubating the mixture with a polypeptide linked to solid support, wherein the polypeptide comprises the protein as listed in Table 1 or Table 2 or a protein comprising a fragment of at least 20 contiguous amino acids thereof; washing unbound components of the mixture from the polypeptide linked to solid surface; and detecting the presence or amount of first antibody bound the polypeptide linked to solid surface, thereby detecting secreted protein from Spiroplasma citri or Candidatus Liberibacter asiaticus. The amount of the first antibody binding to the protein on the solid support will be inversely proportional to the amount of secreted protein in the sample.

In some embodiments, the first antibody is a polyclonal antibody, optionally generated by a method comprising immunizing a mammal with an adjuvant and the secreted protein or an immunogenic fragment thereof and purifying antibody that specifically binds to the secreted protein from blood of the mammal.

In some embodiments, the secreted protein comprises SEQ ID NO:29 or a fragment of at least 20, 30, 40, 50, 70, or 100 contiguous amino acids thereof.

In some embodiments, the citrus plant is not artificially infected or graft-inoculated with a bacterial pathogen.

In some embodiments, the method of detecting comprises detecting the first antibody by contacting a labeled secondary antibody to the first antibody bound the polypeptide linked to solid surface, washing away unbound secondary antibody, and determining an amount of secondary antibody specifically binding to the first antibody.

In some embodiments, the solid support is a well in a microtiter dish.

In some embodiments, the secreted protein is detected and wherein the method further comprises destroying the plant.

Also provided is a kit for detecting citrus stubborn disease or detecting citrus greening disease (Huanglongbing or HLB), the kit comprising a protein from Table 1 or Table 2 or a polypeptide comprising a fragment of at least 20, 30, 40, 50, 70, or 100 contiguous amino acids thereof, wherein the protein is linked to a solid support. In some embodiments, the protein comprises SEQ ID NO:29 or a fragment of at least 20, 30, 40, 50, 70, or 100 contiguous amino acids thereof.

In some embodiments, the kit further comprises a first antibody that specifically binds to the secreted protein.

In some embodiments, the first antibody is a polyclonal antibody, optionally generated by a method comprising immunizing a mammal with an adjuvant and the secreted protein or an immunogenic fragment thereof and purifying antibody that specifically binds to the secreted protein from blood of the mammal.

In some embodiments, the kit further comprises a secondary antibody that binds to the first antibody.

In some embodiments, the solid support is a well in a microtiter dish.

### BRIEF DESCRIPTION OF THE DRAWINGS

FIGS. 1A and 1B show Western blot analysis of ScCCPP1 protein encoded by CAK98563 and spiralin protein in S. citri cells alone and S. citri cells in phloem extracts. FIG. 1A shows that the secreted protein is present in S. citri cells alone and S. citri cells in phloem extracts. FIG. 1B shows that spiralin is present in all samples containing S. citri.

FIG. 2 illustrates Western blot analysis of ScCCPP1 protein in S. citri cells, grafting-inoculated plant tissue and CSD-infected trees from the field.

FIG. 3A illustrates that the anti-ScCCPP1 antibody specifically detects S. citri using leaf petiole imprints. FIG. 3B shows that spiralin protein was not detected in the same samples using the spiralin antibody of FIG. 1B.

FIGS. 4A and 4B illustrate one embodiment of the method described herein. FIG. 4A shows an exemplary membrane-based immunoassay or imprinting assay performed on samples harvested from the field. FIG. 4B shows that the imprinting assay is more sensitive than quantitative PCR for detecting CSD.

FIG. 5. Standard curves showing specific binding of anti-SDE1 antibody with SDE1 using a competitive ELISA protocol. Purified SDE1 proteins were diluted in healthy citrus extract (blue) or 1×PBS buffer (black). Lower OD readings at 450 nm indicate higher amount of the antigen binding to the antibody.

FIG. 6. Results from an ELISA test using the competitive ELISA protocol on freeze-dried tissues from field citrus trees in Texas. The S/H ratio of OD450 from each sample was generated by comparing absorbance at 450 nm from an unknown sample or “Suspect” with that from a healthy control or “Healthy”. The S/H value from a healthy tree is(in blue. Samples in dark green were from tissues without HLB disease symptoms and showed negative results by qPCR. Samples in orange were from tissues with HLB disease symptoms and showed positive results by qPCR. Error bars are standard errors from three replicates.

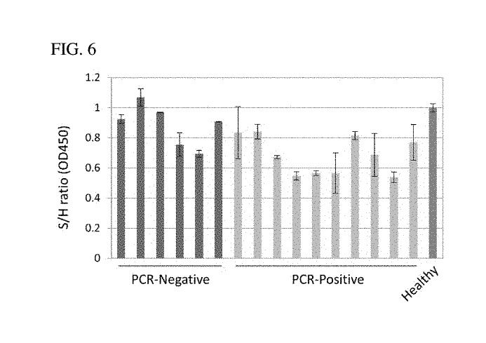

FIG. 7. A Receiver Operating Characteristic (ROC) curve was generated based on the comparison of ELISA and qPCR results of 108 samples in order to determine a cutoff value for the competitive ELISA method. Based on this curve, a cutoff value of S/H ratio of OD450 was determined to be 0.9158, which means that any samples that gave a S/H ratio lower than 0.9158 would be called positive for HLB. Under this cutoff, the sensitivity of the method is 0.857 and the specificity is 0.575. The relatively low specificity value is at least partly due to the known false negative diagnosis sometime observed in qPCR.

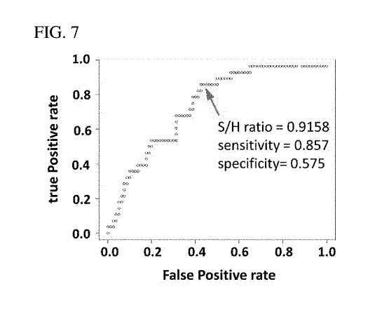

FIG. 8. Scheme of a blind test using 108 samples from Texas field trees. The samples were received as freeze-dried midrib tissues with unknown disease status. The tissues were grinded into powers and the same extracts were used for both qPCR and ELISA tests. Green check marks indicate matches of results obtained from both diagnoses. Comparing to qPCR results, ELISA gave four False Negative (FN) results (4 out of 36; 11.1%). However, 10 of the PCR-negative samples were collected from trees that were previously tested positive by qPCR and these were tested positive by ELISA. Therefore, qPCR diagnosis for these 10 samples was likely incorrect whereas the ELISA diagnosis was correct. The other 16 samples that were tested negative by qPCR, but positive by ELISA may be putative False Positives (FP?).

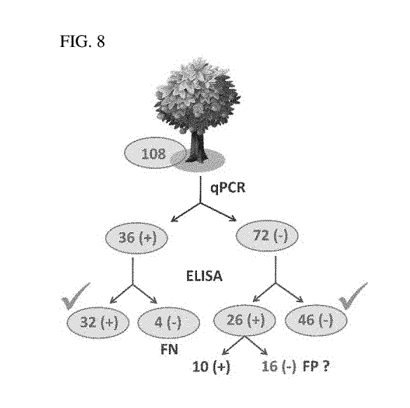

### DETAILED DESCRIPTION OF THE INVENTION

### I. Introduction

Methods for serologically diagnosing CSD using S. citri-specific secreted protein or HLB using C. liberibacter-specific secreted protein in a sample are provided. Bacterial pathogens secrete effector proteins into their hosts during infection. These effectors are usually unique for specific pathogen species or even subspecies. It has been discovered that secreted effector proteins can be used as detection markers for diagnosis of bacterial diseases. Antibodies generated to specifically recognize suitable effector proteins can be used to develop serological detection methods such as enzyme-linked immunosorbent assay (ELISA) and imprint detection. The method described herein is particularly efficient for detecting disease in trees where detection of pathogens using nucleic acid-based methods, such as polymerase chain reaction, is challenging due to the uneven distribution of the pathogens in the infected trees. Furthermore, many bacterial pathogens reside in plant transportation systems, i.e. xylem and phloem; therefore, effectors secreted from the pathogens can be dispersed through nutrient and water transportation.

The method described below is advantageous over other detection methods such as nucleic acid-based detections in two ways: 1) it can overcome the problem of erratic distribution and low titer of the pathogens in the host plant; 2) antibody-based serological detection methods allow rapid and economical processing of a large amount of samples in applications like field surveys.

### II. Definitions

As used herein and in the appended claims, the singular forms “a,” “and,” and “the” include plural referents unless the context clearly dictates otherwise. Thus, for example, reference to “a plant” includes a plurality of such plants and reference to “the tree” includes reference to one or more trees known to those skilled in the art, and so forth.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

The phrase “substantially identical,” used in the context of two nucleic acids or polypeptides, refers to a sequence that has at least 60% sequence identity with a reference sequence. Alternatively, percent identity can be any integer from 60% to 100%. Some embodiments include at least: 60%, 65%, 70%, 75%, 80%, 85%, 90%, 91%, 92%, 93%, 94% 95%, 96%, 97%, 98%, or 99%, compared to a reference sequence using the programs described herein; preferably BLAST using standard parameters, as described below.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10 ⁻²⁰.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is the only natural codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).
   (see, e.g., Creighton, Proteins (1984)).

The phrase “specifically binds,” when used in the context of describing a binding relationship of a particular molecule to a protein or peptide, refers to a binding reaction that is determinative of the presence of the protein in a heterogeneous population of proteins and other biologics. Thus, under designated binding assay conditions, the specified binding agent (e.g., an antibody) binds to a particular protein at least two times the background and does not substantially bind in a significant amount to other proteins present in the sample. Specific binding of an antibody under such conditions may require an antibody that is selected for its specificity for a particular protein or a protein but not its similar “sister” proteins. A variety of immunoassay formats may be used to select antibodies specifically immunoreactive with a particular protein or in a particular form. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective binding reaction will be at least twice background signal or noise and more typically more than 10 to 100 times background.

A “label,” “detectable label,” or “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins that can be made detectable, e.g., by incorporating a radioactive component into the peptide or used to detect antibodies specifically reactive with the peptide. Typically a detectable label is attached to a molecule (e.g., antibody) with defined binding characteristics (e.g., a polypeptide with a known binding specificity), so as to allow the presence of the molecule (and therefore its binding target) to be readily detectable.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same. The class of plants that can be used in the methods of the invention includes angiosperms (monocotyledonous and dicotyledonous plants), gymnosperms, ferns, bryophytes, and multicellular and unicellular algae. It includes plants of a variety of ploidy levels, including aneuploid, polyploid, diploid, haploid, and hemizygous. In some embodiments, the secreted protein is detected in a biological sample from a citrus plant. For example, the biological sample can comprise fluid or sap from bark, fruit, a leaf, a leaf petriole, a branch, a twig, or other tissue from an infected or control plant. In some embodiments, the citrus plant is an orange tree, a lemon tree, a lime tree, or a grapefruit tree. In one embodiment, the citrus plant is a navel orange, Valencia orange, sweet orange, mandarin orange, or sour orange. In one embodiment, the citrus plant is a lemon tree. In one embodiment, the citrus plant is a lime tree. In some embodiments, the plant is a relative of a citrus plant, such as orange jasmine, limeberry, and trifoliate orange.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody or its functional equivalent will be most critical in specificity and affinity of binding. See Paul, Fundamental Immunology.

### III. Detailed Description of Embodiments

Herein is provided a serological detection method for monitoring the effectors secreted from the pathogens into the phloem, wherein the pathogen secreted proteins are markers for citrus stubborn disease. The method includes using an antibody to detect a secreted protein of the bacterial pathogen Spiroplasma citri. Because the causal pathogens Candidatus Liberibacter and Spiroplasma citri reside in the phloem of infected trees, secreted proteins are dispersed throughout the tree along with the transportation flow in the host vascular system. Therefore, although the pathogens cells have a restricted and sporadic distribution pattern, the pathogen proteins are not restricted to the infection sites. This allows for robust detection and overcomes the difficulty from the uneven distribution of the pathogen cells in infected trees. Furthermore, direct detection of pathogen-associated patterns are more reliable and highly selectivity, especially compared to detections of host changes, which may not be specific to a particular disease.

Accordingly, in some embodiments, methods of detecting citrus stubborn disease or citrus greening disease, or the presence of the causative agents, Spiroplasma citri, and Candidatus Liberibacter asiaticus, respectively, by detecting one or more secreted protein from the causative agents are provided.

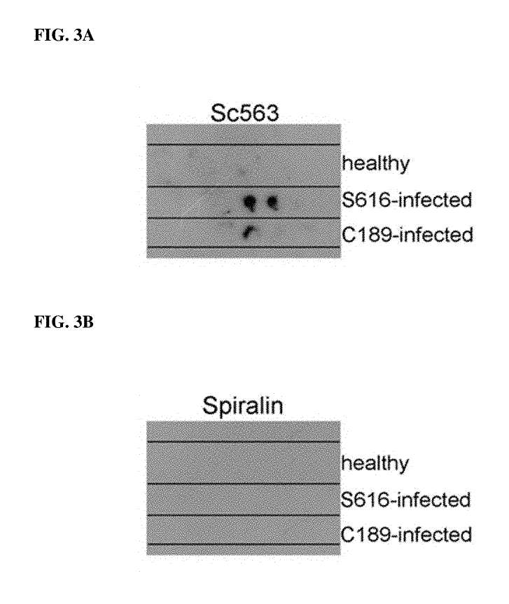

Surprisingly, it has been discovered that the presence of citrus stubborn disease can be detected by detecting a secreted protein as described herein in untreated (unpurified) sap from infected citrus, even trees that are naturally infected and thus have a lower titer of bacteria than an artificially-infected tree. This is particularly surprising as spiralin, a highly-expressed protein from Spiroplasma citri cannot be detected from unpurified sap (see, e.g., FIG. 3B). Accordingly, in some embodiments, the sample of the methods includes fluid of the vascular system (e.g., sap) from a plant located in the field. In some embodiments, a sample is obtained from a plant and is not processed to separate, isolated or purify the secreted protein of interest from other proteins of the sample. For example, the sample is not subjected to extraction, electrophoresis (e.g., polyacrylamide gel electrophoresis, isoelectric focusing electrophoresis), chromatography (e.g., size chromatography, affinity chromatography), or magnetic bead separation prior to detecting the presence of the secreted protein of interest.

Further, in some embodiments, the plant that does not comprise grafting-inoculated citrus tissue or other artificially-inoculated tissue. In some instances, the sample is from phloem-rich tissue.

The methods described herein can be used to detect a secreted protein that is not abundant on S. citri cells. For instance, the secreted protein can be present at a lower level than spiralin which has been shown to be the most abundant protein at the membrane of S. citri cells (see, Duret et al., Appl. Environ. Microbiol., 69:6225-6234 (2003)). Detection of the presence of the secreted protein in a plant sample indicates that the plant has citrus stubborn disease.

In some embodiments, the secreted protein is detected with an antibody. The antibody can recognize (specifically bind) a secreted protein from S. citri or Candidatus Liberibacter, wherein the secreted protein can indicated CSD or HLB, respectively.

Antibody reagents can be used in assays to detect the presence of, or protein expression levels, for the at least one secreted protein in a citrus sample using any of a number of immunoassays known to those skilled in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. Gosling (2000) describes: “Rabbits, hamsters, guinea pigs and chickens are the most commonly used animals for polyclonal antibody production in the laboratory. . . . Sheep, pigs, donkeys and horses are generally only used when large volumes of serum are required and the choice of species is usually dictated by the availability of suitable facilities and by cost . . . . There are a wide variety of standard immunization protocols and many are designed to maximize the response of the immune system to a particular type of antigen or to influence the properties of the antibodies generated . . . . Adjuvants are generally essential components in immunization protocols and they are particularly necessary when immunizing with soluble proteins . . . . A common immunization protocol for both rabbits and mice involves a primary immunization with complete Freund's adjuvant, followed by a number of boosts with Freund's incomplete adjuvant given at intervals of 2-4 weeks until the response is optimum.” A variety of immunoassay techniques, including competitive and non-competitive immunoassays, can be used. See, e.g., Self et al., Curr. Opin. Biotechnol., 7:60-65 (1996). In some embodiments, a competitive immunoassay comprises linking (covalently or non-covalently) a protein to a solid support and then contacting the protein with a mixture of an antibody that binds the protein and a sample. If there is target protein in the sample, then a quantity of the antibody will be bound to target protein in the sample and so will not be available to bind to the protein linked to the solid support. One can quantify the amount of antibody binding the protein on the solid support following wash of unbound components. The amount of antibody bound to the protein on the solid support is inversely proportional to the amount of target protein in the sample. In some embodiments the protein linked to the solid support is a polypeptide comprising a protein of Table 1 or Table 2 or a fragment of at least 20, 30, 40, 50, 70, or 100 contiguous amino acids of a protein of Table 1 or Table 2. The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in the present invention. See, e.g., Rongen et al., J. Immunol. Methods, 204:105-133 (1997). In addition, nephelometry assays, in which the formation of protein/antibody complexes results in increased light scatter that is converted to a peak rate signal as a function of the protein concentration, are suitable for use in the methods of the present invention. Nephelometry assays are commercially available from Beckman Coulter (Brea, CA; Kit #449430) and can be performed using a Behring Nephelometer Analyzer (Fink et al., J. Clin. Chem. Clin. Biochem., 27:261-276 (1989)).

In some embodiments, the immunoassay includes a membrane-based immunoassay such as an dot blot or slot blot, wherein the biomolecules (e.g., proteins) in the sample are not first separated by electrophoresis. In such an assay, the sample to be detected is directly applied to a membrane (e.g., PVDF membrane, nylon membrane, nitrocellulose membrane, etc.). Detailed descriptions of membrane-based immunoblotting are found in, e.g., Gallagher, S R. “Unit 8.3 Protein Blotting:Immunoblotting”, Current Protocols Essential Laboratory Techniques, 4:8.3.1-8.3.36 (2010).

Specific immunological binding of the antibody to the protein of interest can be detected directly or indirectly. Direct labels include fluorescent or luminescent tags, metals, dyes, radionuclides, and the like, attached to the antibody. An antibody labeled with iodine-125 (¹²⁵I) can be used. A chemiluminescence assay using a chemiluminescent antibody specific for the nucleic acid is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies or the secreted proteins (e.g. such as found in Table 1 or Table 2 or a fragment thereof specifically recognized by the antibody) can be immobilized onto a variety of solid supports, such as magnetic or chromatographic matrix particles, the surface of an assay plate (e.g., microtiter wells), pieces of a solid substrate material or membrane (e.g., plastic, nylon, paper), in the physical form of sticks, sponges, papers, wells, and the like. An assay strip can be prepared by coating the antibody or a plurality of antibodies in an array on a solid support. This strip can then be dipped into the test sample and processed quickly through washes and detection steps to generate a measurable signal, such as a colored spot.

Comparative proteomic methods including mass spectrometry (MS) can be used to identify secreted proteins from pathogenic bacteria. For instance, secreted protein profiles from S. citri cells cultures with or without induction of citrus ploem extracts can be determined by MS. Genomic sequencing analysis can be performed to identify gene sequences encoding the secreted proteins. Protein sequence analysis can be used to determine the location of signal peptide cleavage sites based on the amino acid sequence. Secreted proteins for use in the method described herein can be identified using sequence analysis and bioinformatic prediction programs in diseased plants. Expression analysis can also be performed to confirm the presence of the secreted proteins. For instance, the probability of the protein being a Sec-secreted protein can be predicted from a protein's N-terminal secretion signal (e.g., a higher value indicates a greater probability).

Exemplary secreted proteins from Spiroplasma citri whose presence in a citrus sample is indicative of the pathogen (or the corresponding disease citrus stubborn disease) include, the protein encoded by Gene ID: CAK98563 or substantially identical variants. The amino acid sequence of CAK98563 (SEQ ID NO:1) is found in Uniprot No. Q14PL6. Additional secreted proteins from Spiroplasma citri include those described in Table 1 or substantially identical variants thereof. In some embodiments, a protein secreted from S. citri, including any one of those of Table 1, can be used in the method described herein as a detection marker for CSD.

TABLE 1
Secreted proteins from Spiroplasma citri

| | | Probability | |
|---|---|---|---|
| | | of N- | |
| | | terminal | Amino acid |
| SEQ ID | | secretion | position of |
| NO: | Protein name | signal | cleavage site |
| 1 | CAK98563 | 0.777 | 23 |
| 2 | CAK99824 | 0.843 | 23 |
| 3 | CAK99227 | 0.998 | 29 |
| 4 | CAK99727 | 0.906 | 30 |
| 5 | CAL0019 | 0.75 | 27 |
| 6 | CAK98956 | 0.99 | 29 |
| 7 | P123-family | | |
| | protein | | |
| | variant A | | |
| 8 | P123-family | | |
| | protein | | |
| | variant B | | |

Exemplary secreted proteins from Candidatus Liberibacter asiaticus whose presence in a citrus sample is indicative of the pathogen (or the corresponding disease citrus greening disease, also known as Huanglongbing or HLB) include those described in Table 2 or substantially identical variants:

TABLE 2
Secreted proteins predicted from Candidatus Liberibacter asiaticus

| | | | | Proba- |
|---|---|---|---|---|
| | | | | bility |
| | | MW | | of N- |
| SEQ | | (kD) of | | terminal |
| ID | | mature | | secretion |
| NO: | Protein Name | protein | Protein function | signal |
| 9 | CLIBASIA_00460 | 9 | hypothetical protein | 0.667 |
| 10 | CLIBASIA_00995 | 35 | porin outer | 0.55 |
| | | | membrane protein | |
| 11 | CLIBASIA_01135 | 33 | glycine betaine ABC | 0.718 |
| | | | transporter | |
| 12 | CLIBASIA_01295 | 24 | flagellar L-ring | 0.637 |
| | | | protein | |
| 13 | CLIBASIA_01300 | 17 | hypothetical protein | 0.546 |
| 14 | CLIBASIA_01600 | 35 | carboxypeptidase | 0.816 |
| 15 | CLIBASIA_03230 | 16 | hypothetical protein | 0.705 |
| 16 | CLIBASIA_03070 | 49 | pilus assembly | 0.723 |
| | | | protein | |
| 17 | CLIBASIA_02610 | 45 | iron-regulated protein | 0.462 |
| 18 | CLIBASIA_02470 | 13 | putative secreted | 0.452 |
| | | | protein | |
| 19 | CLIBASIA_02425 | 19 | outer membrane | 0.884 |
| | | | protein | |
| 20 | CLIBASIA_02250 | 20 | extracellular solute- | 0.55 |
| | | | binding protein | |
| 21 | CLIBASIA_02145 | 21 | hypothetical protein | 0.746 |
| 22 | CLIBASIA_02120 | 31 | periplasmic solute | 0.721 |
| | | | binding protein | |
| 23 | CLIBASIA_04025 | 9 | hypothetical protein | 0.501 |
| 24 | CLIBASIA_04040 | 15 | hypothetical protein | 0.681 |
| 25 | CLIBASIA_04170 | 28 | rare lipoprotein A | 0.56 |
| 26 | CLIBASIA_04520 | 33 | hypothetical protein | 0.603 |
| 27 | CLIBASIA_04580 | 10 | hypothetical protein | 0.795 |
| 28 | CLIBASIA_05115 | 17 | hypothetical protein | 0.664 |
| 29 | CLIBASIA_05315 | 14 | hypothetical protein | 0.706 |
| | | | (also referred to | |
| | | | herein as “SDE1”) | |
| 30 | CLIBASIA_00100 | 15 | ABC transporter | 0.588 |
| | | | protein | |
| 31 | CLIBASIA_02075 | 44 | chemotaxis protein | 0.624 |
| 32 | CLIBASIA_03120 | 4 | hypothetical protein | 0.65 |
| 33 | CLIBASIA_04560 | 19 | hypothetical protein | 0.57 |
| 34 | CLIBASIA_05640 | 5 | hypothetical protein | 0.668 |
| 35 | CLIBASIA_05320 | 7 | hypothetical protein | 0.83 |

In some embodiments, a protein secreted from S. citri, including any one of those of Table 1, can be used in the method described herein as a detection marker for CSD.

Also provided are kits, e.g., for use in diagnostic and research applications. The kits can comprise any or all of the reagents to perform the methods described herein. The kits can also comprise a scalpel, razor blade or other implement for obtaining a plant or sap sample from a citrus tree. In addition, the kits can include instructional materials containing directions (i.e., protocols) for the practice of the methods. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### IV. Examples

### Example 1

This example illustrates a method for serological diagnosis of CSD using a S. citri-specific secreted protein ScCCPP1 which is encoded by CAK98563.

In the study, scCCPP1 was identified using mass spectrometry of the supernatant portion of a bacterial culture of S. citri. The protein was determined to be in relatively high abundance. Using an antibody generated against ScCCPP1 as the antigen, specific signals from S. citri-infected trees were detected using a direct tissue imprint assay. The results demonstrate that this method is suitable for field surveys.

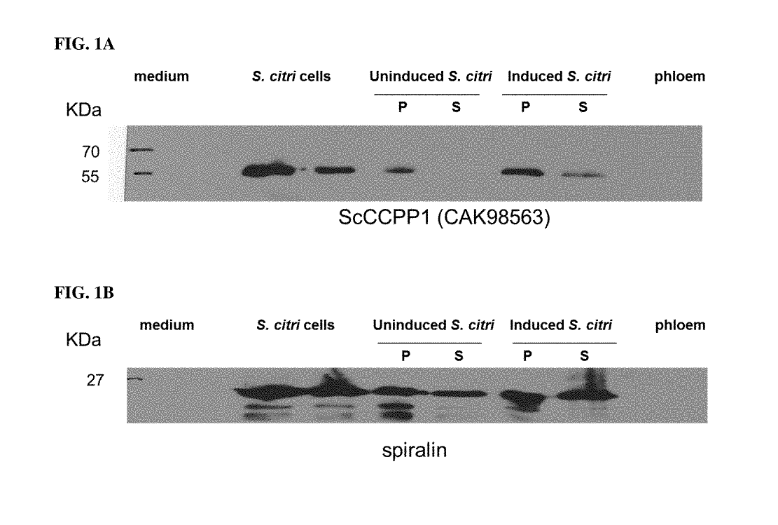

Western blot analysis with rabbit polyclonal anti-ScCCPP1 antibody showed that the secreted pathogen protein was present in the total protein extracts harvested from S. citri cells. ScCCPP1 was also detect uninduced S. citri cells in phloem extracts, as well as induced S. citri cells in either phloem extracts or sucrose only (FIG. 1A). For comparison, spiralin protein which is a major membrane protein of Spiroplasma citri was detected at high levels in the cells including those uninduced and induced (FIG. 1B).

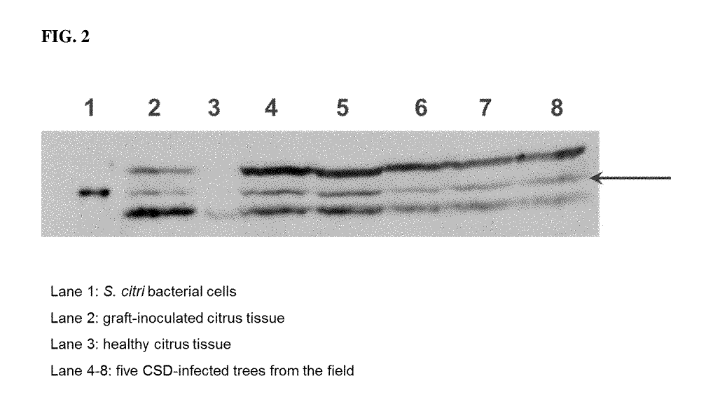

Western blot analysis, as shown in FIG. 2, indicated that ScCCPP1 protein was detected in a protein extract sample from a citrus seedling that was graft-inoculated with Spiroplasma citri (lane 2) or extract samples from CSD-infected adult trees from the field (lane 4-8). Protein extract from S. citri cells (lane 1) was used as a positive control. Protein extract sample from a healthy citrus tree (lane 3) was used as a negative control. The arrowhead of FIG. 2 indicates the position of the predicted MW of ScCCPP1.

In a simple imprint assay, samples of process-free sap from leaves or young branches were obtained from S. citri-infected trees. The plant tissues were removed from the adult trees using a razor blade. The samples were “printed” on a nitrocellulose membrane by pressing the freshly cut cross section of leaf petioles or branches on the membrane, which left faint green-colored marks from the sap in the samples. The membranes were then be taken back to the lab and incubated with a rabbit anti-ScCCPP1 polyclonal antibody for one hour at room temperature (about 22° C.). The membranes were washed with a wash solution to remove any unbound primary antibody. A secondary antibody recognizing rabbit antibodies and conjugated with HRP was added to the membranes and incubated for one hour at room temperature (about 22° C.). The membranes were washed with a wash solution to remove any unbound secondary antibody. Detection was performed using a chemilluminescent reagent and standard autoradiography technique.

Citrus trees that were graft-infected with two S. citri strains (S616 and C189) were examined using an antibody against ScCCPP1 or an antibody against spiralin. ScCCPP1 was detected in imprinted leaf petiole samples from S616-infected trees and C189-infected trees (FIG. 3A). Surprisingly, the spiralin antibody failed to give positive signals from the same samples (FIG. 3B). The results demonstrate that ScCCPP1 can be used as a detection marker for CSD, even in plant samples that do not contain bacterial pathogen cells.

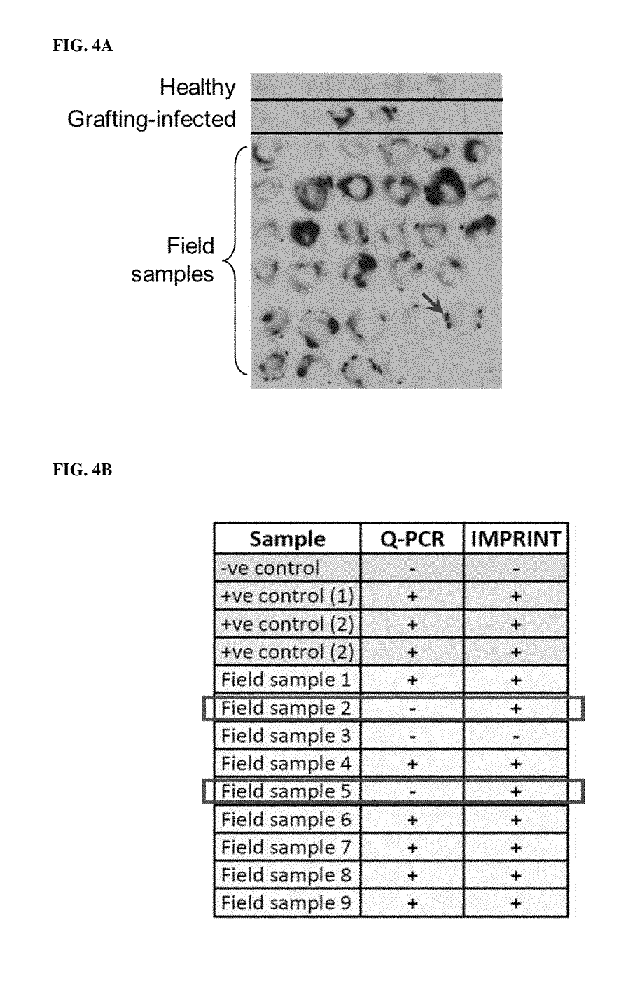

Strong signals of ScCCPP1 were observed in imprints of phloem-rich tissues (e.g., barks) from S. citri-infected citrus trees in the field (FIG. 4A). Four to six different young branches from each tree were printed on the same row of the membrane. Six CSD-infected trees from the field were tested. The arrowhead in FIG. 4 points to a positive signal present exclusively in the regions corresponding to the phloem-rich tissues (e.g., barks) of a branch. This represents the location of the bacterium and the secreted protein. Positive signal was not seen in all tested branches, thus indicating the sporadic distribution of the disease.

Comparison of the imprint method to a nucleic acid-based quantitative PCR (Q-PCR) assay showed that the imprinting immunoassay provides more consistent and more robust detection of CSD infection (FIG. 4B). Young branches from healthy (negative control), graft-inoculated (positive control), and nine field trees were tested by Q-PCR and imprinting immunoassay. Field sample 2 and 5 appeared to be negative for CAK98563 and positive for spiralin by Q-PCR, indicating that the pathogen was absent from these samples. However, the imprint results show that the same samples were actually positive for ScCCPP1 and thus CSD. The data demonstrate that the imprint assay using the ScCCPP1 antibody provides more consistent and robust detection compared to quantitative PCR.

The results also show that the positive signals were mainly present from the regions corresponding to the phloem-rich tissues. This is consistent with the primary location of the antigen. Remarkably, the anti-ScCCPP1 polyclonal antibody gave more reliable and consistent results than an antibody against spiralin protein, which is a major cell membrane component of the bacterial pathogen and one of the most abundant proteins of present in S. citri. These data demonstrate that the presence of CAK98563 indicates citrus stubborn disease in plants even in the absence of S. citri cells. The results also confirm that pathogen secreted proteins can be used as detection markers for phloem-limited bacterial diseases of citrus.

### Example 2

### Competitive Indirect ELISA for HLB Detection Protocol

1-SAMPLE PROCESSING. Approximately 0.2 g of freeze-dried citrus tissues (bark of young branches or midrib of leaves) is used for each test (each sample is tested in triplicate). The tissues are cut into small pieces using blades and ground into fine powder in liquid nitrogen. The powder is then aliquoted in 1.5 mL tubes with 0.05 g sample in each tube.

2-PLATE COATING. Coat 96-well plates (Immulon® 2 HB Flat Bottom MicroTiter® Plates, Thermo Fisher Scientific Inc.) with 100 μL of 1 μg/mL purified SDE1 (CLIBASIA_05315) protein [SEQ ID NO:29] (in 1× PBS* buffer) in each well. Seal the plate with an adhesive plate sealing film and incubate for 4 hours at 37° C. or overnight at 4° C. in the dark with shaking.

3-WASHING. Use a plate washer (HydroFlex™ microplate washer, Tecan, USA), wash the plate with the washing buffer* (300 μL, per well, orbital shake at medium speed for 1 minute) for 4 times. Tap out remaining liquid on paper tower.

4-BLOCKING. Add 200 μL of blocking buffer* to each well. Seal the plate and incubate at 37° C. for one hour or at room temperature for 2 hours in the dark.

5-WASHING. Washing the wells as described in Step #3.

6-SAMPLE PREPARATION. While the plate is in the blocking step (#4), suspend the citrus tissue powder in 1 mL of 1× PBS buffer, and mix by a vortex for 15 seconds. Incubate the suspension on ice for 10 minutes and then vortex again. Centrifuge at 10,000 rpm for 5 minutes at 4° C. Collect the supernatant in a clean tube. Keep it on ice.

7-PRIMARY ANTIBODY PREPARATION. The primary antibody of anti-SDE1 is diluted in RX buffer* with a final concentration of 100 ng/mL. Mix equal volume of plant extract prepared in Step #6 and the antibody solution. Incubate for 30 minutes at room temperature, preferably with shaking. Include positive (purified antigen spiked in healthy citrus extract or HLB-infected tissue if available) and negative controls (1× PBS buffer and healthy citrus tissue).

8-COMPETITIVE SYSTEM INCUBATION. Add 100 μL of plant sample-primary antibody mixture prepared in Step #7 to each well prepared in Step #5. Each sample is tested in triplets (i.e. 3 wells per sample). Seal the plate and incubate at room temperature for one hour or at 4° C. for overnight with shaking.

9-WASHING. Washing the wells as described in Step #3.

10-SECONDARY ANTIBODY INCUBATION. Dilute the secondary antibody (goat-anti-rabbit IgG horseradish peroxidase (HRP)) to 1/5000 in the RX* buffer. Add 100 μL of the diluted antibody to each well. Seal the plate and incubate at 37° C. for one hour with shaking.

11-WASHING. Washing the wells as described in Step #3.

12-SIGNAL DETECTION. One-step Ultra 3,3′,5,5′-Tetramethylbenzidine (TMB)-ELISA Reagent (Thermoscientific TMB Substrate Reagent, Cat. No. 34028, 34029) is used to detect positive signals. Bring the substrate to room temperature prior to use. Add 50 μL of the substrate into each well. Incubate at room temperature for 1-5 minutes or until color development, and then add 50 μL of the Stop Solution* to stop the color reaction. Read the optical density (OD) of each well at 450 nm using a microplate reader (Tecan M200Pro).

13-DATA ANALYSIS. Take the averaged OD of the three repeats from each sample (S) and calculate a ratio compared to the average OD of the healthy citrus tissue control (H).

### Buffers:

*1×Phosphate Buffered Saline (PBS): 80.0 g NaCl, 14.4 g Na₂HPO₄, 2.4 g KH₂PO₄, 2.0 g KCl Add ddH₂O up to 10 L, pH=7.4

*Wash Buffer (1× PBS with 0.1% Tween-20)

*Blocking Buffer (3% non-fat milk in 1×PBS)

*RX Buffer (1× PBS, 0.1% Tween-20, 3% non-fat milk)

*Stop Solution (2M H₂SO₄)

### Results

Binding of Anti-SDE1 with SDE1 In Vitro

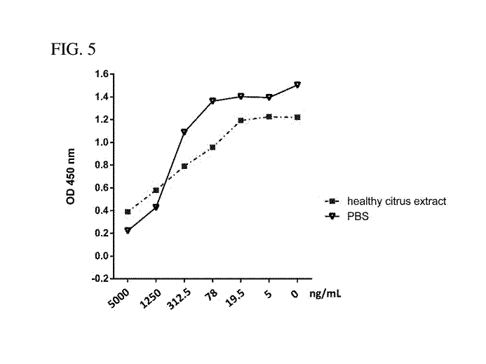

The sensitivity of this ELISA protocol was determined by calibration curves using purified SDE1 antigen. A serial dilutions of purified SDE1 proteins (5000, 1250, 312.5, 78, 19.5, 5 and 0 ng/mL) were spiked into either 1× PBS or healthy citrus extract (Rio Red grapefruit) and OD450 was determined in each sample (FIG. 1).The results show an inverse correlation between OD450readings and the concentrations of SDE1 in both 1× PBS buffer and citrus extract, demonstrating a specific binding of anti-SDE1 antibody and SDE1. See, FIG. 5.

Evaluation of the Competitive ELISA Protocol for HLB Detection using Citrus Samples

The competitive ELISA protocol was tested using field citrus samples from Texas. We received freeze-dried midrib tissues from trees grouped into two groups (10 positive samples and 5 negative samples) based on HLB symptoms and diagnostic results from Taqman qPCR (Li et al., 2006).

Our results from competitive ELISA show that the average of S/H ratio for the positive samples was 0.60, whereas that of the negative samples is 0.88 (FIG. 2). These values are significantly different as shown by statistical analysis (P value=0.0035), suggesting that the competitive ELISA method was able to detect HLB from naturally-infected citrus samples.

Note that we found two “negative” samples that showed relatively low S/H ratios, which indicate they might be infected. Indeed, being called “negative” may not necessarily mean that these trees were actually free of the HLB-associated bacteria. It has been observed multiple times that qPCR-based diagnosis, which detects specific DNA sequence from HLB-associated bacterium, did not provide correct diagnosis for all infected trees due to the uneven distribution of the bacterial cells in infected trees, especially at the early infection stage when the bacterial titer is low and the trees do not exhibit disease symptoms.

Comparison of the Competitive ELISA Method with qPCR

We further applied the ELISA method in a larger scale test using 108 samples with the goal of comparing results obtained from the qPCR-based diagnosis. These samples were collected by our colleagues in Texas and we tested them simultaneously by TaqMan qPCR and the competitive ELISA protocol.

We first conducted a statistical analysis using the dataset to generate a cut-off value for putative positive samples based on the S/H ratio of OD450. Based on the diagnosis provided by qPCR (which may or may not be accurate, since this method is known to miss positive samples), we separated samples into known positives and known negatives, and then compared the ELISA values for every sample. A two sample t-test with equal variance was used to determine the difference between the two groups. This analysis showed a significant difference between the two groups, with a P value<0.001. The S/H ratios were squared before applying the t-test to meet the normality assumption. We further calculated the true positive and false positive rates, and used Receiver Operating Characteristic (ROC) curve to determine the cutoffs, which is S/H=0.92, with a sensitivity of 0.86 and a specificity of 0.58 (FIG. 3).

Based on the qPCR, we found 36 positive samples and 72 negative samples. From the 36 qPCR-positive samples, 32 were also detected as positive by ELISA using the cutoff value generated in FIG. 3. However, from the 72 qPCR-negative samples, 26 were also detected as positive by ELISA. See, FIG. 4.

From the comparison, the current ELISA might have 4 false negative, and 26 putative false positive. However, when we compared to previous qPCR results using different tissue collected from the same trees, 10 trees (from the 26 that were ELISA positive but PCR negative) have been shown to be HLB positive, again, using different tissues from the same tree. These results suggest that these 10 trees were HLB-infected, although the tissue collected for testing in this trial probably did not have the bacteria, thus resulting in the (false) negative diagnosis by qPCR. Notably, these tissues contain the secreted protein SDE1, leading to positive results by ELISA.

Taken together, our data showed that the ELISA protocol, by detecting a different biomarker, provide additional and complementary information with qPCR and in some cases could better detect HLB when the tested samples do not have the bacterial cells although the tree might be infected. Therefore, it is a useful detection method that enhances HLB diagnosis.

We also evaluated the ELISA method for HLB detection using fresh citrus tissues in Florida (trial performed in Southern gardens company, Clewiston, Fla.). We collected leaf midrib tissues from 20 field trees and tested them by both ELISA and qPCR. We found matches in results for 16 samples (9 positive and 7 negative), one false negative, and three potential false positive (Table 3). Again, the three samples that showed negative results in qPCR but positive results in ELISA may not mean that the ELISA assay gave false positive diagnosis. HLB infection rate in Florida citrus trees is estimated to be >90%. It is likely that the three PCR-negative trees are actually infected, but the tissues that were collected for testing did not have the HLB-associated bacterium. This does not affect the ELISA test because the biomarker, i.e. SDE1, is a secreted protein and its location in infected trees is independent on the location of the bacterial cells.

Table 3 depicts a comparison of ELISA and qPCR diagnosis of HLB using 20 fresh citrus tissues in Florida. Comparison of ELISA and qPCR results showed matches in 16 samples (highlighted in green). One sample was tested PCR positive but ELISA negative, which indicates a False Negative (FN) for ELISA. Three samples were tested PCR negative but ELISA positive. These may potential be False Positives (FP?) for ELISA, but could also be due to False Negatives of qPCR.

TABLE 3
A comparison of ELISA and qPCR diagnosis of HLB

| sample | qPCR diagnosis | ELISA diagnosis |
|---|---|---|
| R2T26 | + | − (FN) |
| R2T42 | − | − |
| R2T58 | − | − |
| R2T60 | + | + |
| R2T75 | + | + |
| R2T88 | − | + (FP?) |
| R2T90 | + | + |
| R2T103 | + | + |
| R2T106 | + | + |
| R2T107 | − | − |
| R2T108 | − | − |
| R2T119 | + | + |
| R2T121 | − | − |
| R2T122 | + | + |
| R2T123 | + | + |
| R2T124 | − | + (FP?) |
| R2T135 | + | + |
| R2T138 | − | − |
| R3T25 | − | + (FP?) |
| R3T27 | − | − |

All publications, patents, accession numbers, and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

CAK98563 protein from Spiroplasma citri (411 aa; 46,015 Da)
SEQ ID NO: 1
MRKLLSIFAATTLVTTSAASAVACSGAPQGNLIPIFMYNGNQKFSHAPTVTRKSINGIDDVTQS
GKDENGAPYEYSLQGGRMGLINGLINNAINPILNGINLTKDNSATTGKGAKWTDEQIAAGLEGQ
KEQLVQTAKTDAQDPFNSSKKINQKAIWKDLFNNYSTSFDSSYSQVAFLANENKAILNKTNDNL
VTMTGNAEKTNNKNWVKEHTWPDGKKSPYTPSSLKVLSPIASILEWFNDPKNSYNQGYNQIDQN
RGYQSARYLAIAIPNVTIRFEFQGEHNCFTFTVTIDKLVAYANYLVYENPNSTKDNPSYGHQWF
FLSYGFYDFESLKDDDYHHYNFNAIPDDVKIDKNIKVALGFFKKNDDKGILTADEDKEVGKRGQ
FPTAETDYTFPALKWKINVNSITDQYK
CAK99824 protein from Spiroplasma citri (566 aa; 63,899 Da)
SEQ ID NO: 2
MKKILSILGAMSILSSTGTSIYACTKKDFERFTDPAIVDEVKRWIIAKINSENESNVVKYSFND
IFTKAALKEMTTRLLDTNISKFFYASEEATRARYTGVTIDVNQPDSLLIKQFINFVEQVALDNL
STKYSTGISNSTPLETTIAGQGYAPDYQSEGWYVGGSQSYFWKGTGKPNYMQSRKGQGEANFEI
KSKSAGANYTDFNAMSEDDKKAALKTRFKDYYTHVEIPAVIDKIITATYLHQNEIKRYGSGNNS
SIYLNRNSALFNALQSWDTISGARWKSYIKMVWELKLDKEELDKLFADKGPLANVEKLNSDLTN
NQDILMKILKEMFNAKKDNIFNNMIKDGIDPIFGISGFKGFVGIDKNKNPNDIFTTLNNADSYK
QKVIDTATPGIIKSGEGTDPSSYQFLDTNKRYGSFVLTLPIYAVDLMKNMNINYKNDNKNNKEL
SLTWYGSGGTPTDLDQAWLAQQGGIKRSLSWLYNKKGYLGTYDENGQPLYNDNGTPVDITKNIK
GQILKWIEYTFAKQQNLQTAAKTRLYSLVFANNPENVYSQTLYDAIGSYIIKED
CAK99227 protein from Spiroplasma citri (730 aa; 82,870 Da)
SEQ ID NO: 3
MRKLLNILAAATLAATPALTASCKTKAKSAEDKYKDSSVENLPNGPLKSKILQTTLFTKATIAN
RHENLNTYTPSMLQMLMRLPDSYKDKDGNIVDIDYYRGKYLNKNNGMPLTTLSSNYDYMNLLDN
ELYNTEKQTKKKISDYSDKDPLPSIPNLTKNSNMLNYWYDGGPLSNYSIVKEILPTKCDDKRIN
QNIVDACNKAIFEMPRTTYFYNFNMDYSPMATKDIKFDTDTNQNFIINNQKFAAGGPFKTAQKS
EEQDKLSIILQLISMADMFTDRSQSKTYINQLNKFLALSGDSDGTFMGSILGAIYYQIFASPKL
PNDPTKENANYTFAKLGVTKALQLLRKDSTERQAIKQKIDVFFDAQSQVFSDLLAVRPMQPGLD
LNKPEGQYKNRDAMWNGKTPNLRLVDLLFKKDASTKSLAELFTDFGEYLDDLYTKADVECQEEA
NNSISSFLKLAANVVTPGFKVVLQSMSKMMLSKSEGGLGTLSVNDINRFVVALSKGILQSANAL
TEVSKLPWSEATDQEQNQTKITQLLTGSDDPSTPTKDSFMDLAFTWFNDSTQPVRALLNKLYFD
PDSETRKDLLAINNALYEYSNNLLLGANWNISNGQLEENKLSYDIEYKGTGDADVVANLDLHQN
WYIPKSEIKTYQDLNAAYLNALGNRDLDWFMKYDGLGNNYQKVHYKYKVTWMNINPGDDSHQYW
VISNIQWFAKDISGQWKRYYDAIEND
CAK99727 protein from Spiroplasma citri (291 aa; 33,893 Da)
SEQ ID NO: 4
MKWLLTLFSVFVLGFGSSLGVVSCTVRAKHEPDDNDELDHNQDLEILNQIKKEAKQTLSTWWQT
KTMIDIIKDYQEQISSFKELVTQVKTKNDGSLTLTSIAISKYCFLNQLLIGFKAEFNNLNQHLQ
NRYSNYYVDTMPLFLGENDISFNLYNINFDKIAKLLADTAQAVLGITVQVNIAYEVRFKGLPTE
DNIQVSITTTNDSEVLNNIQDKVENYFVNFLDTIFKAKNYRIISKQFNIKTDIVWPIISKELND
RNISFQHIIFNFLYYGQQIYLIFIPLNIWTLTRLF
CAL00019 protein from Spiroplasma citri (308 aa; 34,572 Da)
SEQ ID NO: 5
MAKIGGKSSIALPTLFEIGGNSPTEKAIGDVAVLQIKKILEADSQYIDATEMMMMSAKQISMED
GFEQGQYVFPERMVWGNDYDSSAGAEQQSVGVRRATVMMDQMLTFKYDVPSFDTVRFMESPVEV
RTNTIGEWMRTITRNWYSNMNAIYLQGVIDSCIATGQYIILPIPTDADSAQQTFYKINDIAINL
VQKINALMFGTKKEDLMVHVAMPAFAQFTKAYTKILDQIAADTLATGQLWRKMIVGVDVFESWY
LGRQFNKGKETGINKDLDFNLNFSQTVGAWGFIGHKEDCAMPQGWKSIQQVN
CAK98956 protein from Spiroplasma citri (373 aa; 41,700 Da)
SEQ ID NO: 6
MKRFFTLLAVLNVATGSTVMVASFTIAQGAHLNPVDSLLLIGKDIDTSKAIDDAKSNLQFTNYY
ILGDSLSDSHGIEKLVKNSFKLDIKIGTNDPNNLENYQNGSLSNGNTAAVLLNAKLGFDKIRPG
IPNDYAGDFGRNYAIGGATAVDVVGTAGMLLNRVTIEKQAQALVSQHKLRSTDLVLFEIGGNDL
FQIIDTTDPQTELELMHQSVERIKIALFTLLNNGIRKILFSDAPNVSAIPRYNNQNTDDTLKKR
ANNISTEFHARVAKMIELANTYYKNAIRNWGLYDNLSVLMTEFKARHPKGNITVNFNNLNLDFI
KIIEEKMLNAQRNPALPANANIDDYFFFDIVHPTREVHQLAMEHYYQTIKEWT
P123 protein variant A from Spiroplasma citri (463 aa;
52,865 Da)
SEQ ID NO: 7
MLRQFGGLIGSNSLTFGCMLKLTDKIKTHYYSATGQDLGTKVYSTIDLGQNRINVNGGTDEIVI
LSQDFQALDKDTPLTNLDNKLDFNPTVDGTLESYVIDLINLQTIGKTNFKITTYSENPFTLNDN
ITAFQNFQGEWQTMAKFIKPNGNVQAWNSVIKTSALDYDLLEETTFFYPQAVLPPDPFTYKPYT
VNILDSNTIKPLKANITAAKQCTSSISFIWQKDWINLFNNKYDNVYYELEVDLKQINTTITTWE
QLLNAYSSIQFNNLNNLQVKCAPPNIENFIPNRNINILGGYSTCENNLGWFSNGKLKVKGDKQI
DKVNFNGSLIYDLANIKENKKTTLFTGETTGLPSGYYQNRYSFSKNCDFSFLLQITKLSDNDDN
KIKELYVQAWHTNDMKLKIRFTKVVFPDFSFYYSDMPVVKLYKYFGNDKNISIDIDNSNILGIR
NDSINPISITFIPRS
P123 protein variant B from Spiroplasma citri (1067aa; 123,
417 Da)
SEQ ID NO: 8
MSRINPSVMEELRRQAHDTLIDIRENIRDNRHWMVFPFNVYSSYIPDTETGWKPNNAKPSDYGS
YTRIFDNMESTIAQWQGSLKIVQKYKGEEINILTIDDFNKQPINSTIWANNETPFAIKLKDENE
NNYWKLIDGENIYDINKMLEYLKQYKLSYFINNDNKADNFYTKEVKVGKYWDAYMIDDRSSIQF
GDAKFEQVFTQQSTERQYTLLILDEDAEKLNINDYYNFYTVFNDRGLNIGGGDINSAPQDRQWL
YPSNVINYYSPYDGSFLWQEITFSSINDKISHSGLSVRQFIQTSAPGEGYCFPKITYDKKLNKG
IVELDEVLLKDPGVRAMAVKFYGNPILFDMPVIGRPIIKGEFNKKVMNSRDLLMYNMYPAPPDK
INTYAAPEVSWYNIQGIQPTMITGYEDWKKDMESRYDFWNQKNSEGIVITDKVTSTATNPTVGT
TINEEDNNSEFYWSKSWKITPPNKVKVNSGSVDNIISTQASSYFRRKDEFGNTVGQVQIEYNKM
GSCNVWDILNINNFINRQITVLPLNYTHKLVFNPFTIPGGVGKFLNFISFGIPWGWVINQDKKT
WPNFQWLNGFMSANVYSFYNDAFWSEKSKGKGYLPFEIFREQGNDKVGAIFGANATSLGFTTLL
TDKCKGTVYTNDGKITEQITYSTYNLKQLNEKTNRIYLINEQTKAVEAQTPVSDSEKDNDCEFL
QTPDGTNCSYIIDMFSIQALYKGNFEIIFYADNPYTNNEEDYLRLSVWSFRGKTKSVLNNYLRD
MTTNYKTSFLLPHDYEIPTFNYPQYVLPPVPYNYQPYTKDIMPAGVVQTLKTKITAPAQCKENF
PPVNLFDDTAEHNAYYETEIDLRQIDPTIQSIDKFKSAYKTIEISNLGNLQVECGPPNIENFIP
NRNVDILGGLNCNINLGSLANITLQESGRSQTDKVNFDNIRTLYTNNITNEVQTFEPIAYTNGL
QGDDYENRYFASDKRVVLDFLLQITKLSNNNDNDKIKETYLQAYYTNDVKLKIRISKIININLL
LDSTMHWKYFGNNKNIVLDVDNPNVLGIRNKSENPVKIIFIPR
CLIBASIA_00460 protein from Candidatus Liberibacter asiaticus
(98 aa)
SEQ ID NO: 9
MRHLILIMLLSILTTNIARAQVYHIHSPRIATKSSIHIKCHSCTLNKHHINKTPSSSSAVYTKK
EELIDGKKAMITTDNFMGGEPIIFIKYLFEEDKK
CLIBASIA_00995 protein from Candidatus Liberibacter asiaticus
(351 aa)
SEQ ID NO: 10
MGLKKFFLGTVAVATIISYSESFAYVRGKTSMVSNNRARNRSAGKVGNVLPHITKVGGSLEKSL
QARYHKLNGNNEFNSLAYDIPVKGNLEVNANAGDVTGVAKLKLAVDDVLSMQFAESDVRALAFT
VPSSKLSVEELSLSMKGARLGYYKSWSDEVNPVYSPTTLYNDARGLDKMMSLSYRHSFGLLKAG
LSTDLLQKDGLKQVLGIGYMASYAIGKIRSTVTGGYDAGTNNVAIRANISSPVSRAGTLDCGAV
WASGDNSYYDKSKYSVFAGYKFDVAKSIIISGGGQYFGDINKTGKDGWSAGISAKYMISSGLEA
QASVAFNDNFVKKGVAIDKGVDLSVGLKKSF
CLIBASIA_01135 protein from Candidatus Liberibacter asiaticus
(309 aa)
SEQ ID NO: 11
MYKILAVCLFLTTFSISYARDADSCTPVRFADTGWTDIAATTAMTSVILEEILGYKTNIKLLAV
PVTFRSLKNKGIDIFMGYWYPSLEKFIAPYLEEGSIKLVAENLQGAKYMLAVNDVGFALGIKSY
QDIAKYKKELGAKIYGIEPGNEGNQRILDMINNNKFSLKGFRLIEASELASFSQIRRDQRNNIP
AVFLSWEPHPINSDLNIHYLPGGEEISGFGEASVYTVVRSDYLDKCPNISRLLKNIKFSVALEN
EMMKLILNNKQDRQFVGRTMLRTHPDLLKNWLIGVTTFDGQDPSRQLERFMNN
CLIBASIA_01295 protein from Candidatus Liberibacter asiaticus
(238 aa)
SEQ ID NO: 12
MYRYSFIVLCYSSLLFGCHSSISEITGIPQMSPMGSSLDENNRMPFLGIDFKNSDSTKKSYSLW
RDSHAALFKDSRALNVGDILTVDIRIDDQAVFDNQTGRSRNNSLHRKLSGGFSLFGQQTPQMNG
NLNYDGGGASSGKGSISRAEKLNLLIAAIVTAILENGNLIISGSQEVRVNDEIRSLNVTGIVRP
QDVDAHNSVSYDKIAEARISYGGKGRTTELLRPPIGHQLIENLSPL
CLIBASIA_01300 protein from Candidatus Liberibacter asiaticus
(175 aa)
SEQ ID NO: 13
MILLPIIYYYKKRDMLSQLLFLLFFFLQGFANQSYGDPTLVDREIQQYCTNVIDSVRERDYLSQ
KKVLEDLQKDIEQRVILLENHKKEYNLWFQKYDSFIMSYNKNILDIYKKMDSDSAALQLEQIDP
DISSHILMRLSPRQSSLIMSKMNPKSATMITNVVANMLKFKKLKRSS
CLIBASIA_01600 protein from Candidatus Liberibacter asiaticus
(336 aa)
SEQ ID NO: 14
MRQFLVIIAMAMLSNIFPFLSIAETNKLPYYTLLDTNTGHVIAENYPDHPWNPASLTKLMTAYV
VFSFLKEKKAMLTTPIIISKNASEYPPSNSTFKKGSTMTLDNALKLLIVKSANDIAVAIAESLC
KTEKKFVQHMNNTSKNLGLSATHFMNAHGVVQHGHYTTARDMAILSWRIKTDFPQYMHYFQIKG
LEIKGKKYPNTNWAVGTFLGADGMKTGFTCASGFNIVASAIQGDQSLIAVILGALDRNTRNKVS
ASLLSMGFYNKTDRKKINYIIKDFYQQNLSNEVPNISEEVCTTQKEIINYNTQMKEAKDKESHF
IDFEKITLLKNKITKK
CLIBASIA_03230 protein from Candidatus Liberibacter asiaticus
(162 aa)
SEQ ID NO: 15
MNFRIAMLISFLASGCVAHALLTKKIESDTDSRHEKATISLSAHDKEGSKHTMNAEFSVPKNDE
KYTISSLTKKIESDTDFRREKATISLSAHDKEGSKHTMNAEFSVPKNDEKYTISACASDDKGNK
STLCVECPSPSTPGQYDLNHCAECENTTSKGLCP
CLIBASIA_03070 protein from Candidatus Liberibacter asiaticus
(474 aa)
SEQ ID NO: 16
MRYLQRTFFTMMSIFLFSSNPSVAKLPPIKEANAAVINISDVEIGKGKKISIGLNKVIILQVPV
DVQDVLVSDPTKADVVVHSPRTMYLFGKNVGQANVILIGHDGKQMLNLDILIERDIAHLEMTLR
RFIADSNIRVEMVSDTVVLHGMVRTIQDSQRAVELSETFLSQSGRNQYANSSSKKVMNLLNIAG
EDQVTLKVTIAEVRRDILKQIGFQHSITGSSSGPSKSFAADFGGKFVSEGGDFSVKGVLDRFSF
ETVLHALERATAIRTLAEPTLTAISGQSASFTSGGQHLYKTVSSSTGATSVTTHDYGVVLHFTP
TVLSPGRIGLRIQTEVSEPVIGVNAGDMPSYRVRKADTTVELPSGGTIVLAGLLKDDIQQLKEG
IPLLSKIPILGALFRNSRFNREETEIFIAATPFLVKPVAMRDLSRPDDHYSVEDDAKAFFFNRV
NKIYGPKEASEVEGQNYKGAIGFIYK
CLIBASIA_02610 protein from Candidatus Liberibacter asiaticus
(412 aa)
SEQ ID NO: 17
MYFITIISIVFTLPSHALSIVPNVSLEKVLQNYATIAHAKYEDALMCARTLDSAIETLVTTPNK
KNLENARLQWIRARIPYQQSEVYRFGNKIVDTWDKKVNAWPLDEGFIDYVDSSYGKENEENNLY
TANIIANSKIIVNEKEIDLSIISPDLLRKLHRANGIDTNITTGYHVIEFLLWGQDLKTNVREPG
NRPYTDFDIGNCTGGHCRRRVEYLKVVSKILVSDLEEMMKAWGPDGQATKDLMKDINAGLNSII
TGMTSLSYNELAGERMNLGLILHDPEQEIDCFSDNTYASYLNDVIGIISSYTGEYIRMNGEKIH
GASIHDLISHNNRNLAQEINDKFSNTMKDFHILKDRAENIESFDQMISENNPEGNKIVRNLIND
LITQTESLRKIRIALDLIEPNRVIGNVP
CLIBASIA_02470 protein from Candidatus Liberibacter asiaticus
(131 aa)
SEQ ID NO: 18
MCRKIIFALTIIAIAFQSMALNCNETLMQADMNQCTGNSFALVKEKLEATYKKVLEKVEKHQRE
LFEKSQMAWEIYRGSECAFAASGAEEGTAQSMIYANCLQGHAIERNEKLESYLTCPEGDLLCPF
INN
CLIBASIA_02425 protein from Candidatus Liberibacter asiaticus
(205 aa)
SEQ ID NO: 19
MQKLFLAVGVSSLALASFCSAQAADPVRRAHHGGRGVVPTIATNRYVPIRHDFNGPYAGLSALY
NGSFGEEAHHNAGGSIFAGYNVEDSCIMYGVEGDVRYTVPVLADNIHSLHGIGGSLRIRGGYEV
SDSLLLYATVGPDVAQKYETGKAGEITPIAIGGTAGVGVEVGGLSESLVARLEYRASKYSKVEG
FYNTISLGVGMKF
CLIBASIA_02250 protein from Candidatus Liberibacter asiaticus
(195 aa)
SEQ ID NO: 20
MFKRTIYTYLLLLCGFTEAFSTENTTKYLTLYTDQNQSVMLPIIHSFEERTGVKISPIYTSSIQ
RPPITQGSPVDVIITKDETSLALNEDLLHKLPAHLIKKNSFVLKNENKKLMRISFDTQVLAYST
KRIKIADLPKSVFDLTNAQWKKRLSIAPNNISFHRLLNTMEQTPNKTVVQDFIKNITANEILTK
YKR
CLIBASIA_02145 protein from Candidatus Liberibacter asiaticus
(210 aa)
SEQ ID NO: 21
MKYRVLLLILFFVFSHAKFANSARFANKVAEFAGMDKITGRVLTFDVEINQSAQFGSLIIKPMV
CYSRDDREAQRIDAFVSISEIFTDRIVRSIFSGWMFADSPAMNAIDHSIYDIWLMQCKDPINDS
ISNSESISKKALSEYSSTDITSQGSEKSSGSSSNKTLEKESSQPLENNLSMDLKGRPIQELGNN
LSDSGLNEQDHNDVQISK
CLIBASIA_02120 protein from Candidatus Liberibacter asiaticus
(294 aa)
SEQ ID NO: 22
MLRYFICLLFSYIPMSASATTQKKVVLSSFSIIGDITQNIAKDLVTVTTLVEAGNDSHSYQVTS
ADAIKIQNADLILCNGLHLEETYMKYFTNLKKGTKIITVTDGINPIGVSEDTSVDSEPNPHAWM
SLTNAMIYIENIRKALTALDPSNAKKYELNAREYSEKIRNSILPLKTRIEKVDPEKRWFVTSEG
CLVYLAEDFGFKSLYLWPINSDSERSPSMMRHAINQMRSHKIKFIFSESTNSDQPAKQVAYETN
ASYGGVLYVDSLSKPDGPAPTYLDLLRFSLTKIVDTLF
CLIBASIA_04025 protein from Candidatus Liberibacter asiaticus
(96 aa)
SEQ ID NO: 23
MTISKNQAILFFITGMILSSCGDTLSDSKQHNKINNTKNHLDLLFPIDDSHNQKPTEKKPNTSS
IKIKNNIIEPQPGPSRWEGGWNGERYVREWER
CLIBASIA_04040 protein from Candidatus Liberibacter asiaticus
(159 aa)
SEQ ID NO: 24
MKRLKYQIILLSLLSTTMASCGQADPVAPPPPQTLAERGKALLDEATQKAAEKAAEAARKAAEQ
AAEAAKKAAEKIIHKDKKKPKENQEVNEVPVAANIEPESQETQQQVINKTTTSQTDAEKTPNEK
RQGTTDGINNQSNATNDPSSKDKIAENTKED
CLIBASIA_04170 protein from Candidatus Liberibacter asiaticus
(276 aa)
SEQ ID NO: 25
MKRFSCDCLLKGSVVCVVVLGMSSCFFSSTYKDDKLEYFPESMYGVTASDRIVSGKRVPRGGGR
YFLGKPYQIMGRWYVPRQYTAYAAVGMASWYGKAFHGRLTANGEVYGTEYITAAHPTLPLPSYV
RVTNMENGISLVVRVNDRGPYHSNRLIDLSNAAAKILRVEERGVSKVHVEYLGMALLNGMDQEY
LRSTVMVNSATVLPLGCQYREEIVVIPYLLTRSRTVHLNNCDDDSLQKQREISLRERKKSNLIP
LPNGYSPPRKMGKIPIPSRF
CLIBASIA_04520 protein from Candidatus Liberibacter asiaticus
(304 aa)
SEQ ID NO: 26
MIRKYVLALVFFLVPCTASVAQKVRLVSWNINTLSEQEGVSLWKNSVKRTTSDYTLLRQYAKNL
DADIVFLQEMGSYNAVAKVFPKNTWCIFYSTERLINHSKRDSNNDIHTAIAVRKKNVRVLQQSY
PLLGAKDSFSRAGNRRAVELLVEINGKKIWVLDIHLKSFCFLDSLENTYSPSCSLLSQQAQWLK
DWITQKKESLVPFVIAGDFNRKINYLGNNDDFWKTIDPNDSLIRFPKEKDSRCNANKNLRNKIP
IDYFVMDQNAYKFLIQESFSEILYNEDDIKSRGKRLSDHCPISIDYDF
CLIBASIA_04580 protein from Candidatus Liberibacter asiaticus
(116 aa)
SEQ ID NO: 27
MFWIAKKFFWISVLLIVLSNVYAQPFLEETEKGKKTEITDFMTATSGTVGYASNLCNAKPEICL
LWKKIMRNVKRHTLNGAKIVYGFAKSALEKNERESVAIHSKNEYPPPLPSHH
CLIBASIA_05115 protein from Candidatus Liberibacter asiaticus
(185 aa)
SEQ ID NO: 28
MFLNVLKDFFVPRIRFLIVLMVSSVSAGYANASQPEPTLRNQFSRWSVYVYPDLNKKLCFSLSV
PVTVEPLEGVRHGVNFFIISLKKEENSAYVSELVMDYPLDEEEMVSLEVKGKNASGTIFKMKSY
NNRAAFEKRSQDTVLIEEMKRGKELVVSAKSKRGTNTRYIYSLIGLSDSLADIRKCN
CLIBASIA_05315 protein from Candidatus Liberibacter asiaticus
(154 aa)
SEQ ID NO: 29
MRKNLLTSTSSLMFFFLSSGYALSGSSFGCCGEFKKKASSPRIHMRPFTKSSPYNNSVSNTVNN
TPRVPDVSEMNSSRGSAPQSHVNVSSPHYKHEYSSSSASSSTHASPPPHFEQKHISRTRIDSSP
PPGHIDPHPDHIRNTLALHRKMLEQS
CLIBASIA_00100 protein from Candidatus Liberibacter asiaticus
(208 aa)
SEQ ID NO: 30
MDKKLNKIIKKKIISCSLAVLLCTSLSSCFFHNNPVNIYDLTESTKYDESVQRHIQLIITEPIT
EKILNSEDIIVRSSPIEIQYLIGSQWSDKLPRMIQLKLIANFENNGKISTVVKPNQGIYADYQI
ISAIRSFEINIDRHCAIITMSLKIINAHDNSLVGQKVFHVEEKLEKDNKLHFIQSLNRAFSRIS
SEIIDWTLSSLPLSDN
CLIBASIA_02075 protein from Candidatus Liberibacter asiaticus
(396 aa)
SEQ ID NO: 31
MNQKYLICTMMVAMDVFFSFATDQDLVRTIVPYQCVRSLQRALDEAMRGDISLQKKIPDIVKET
GVQLRATHMDVFVDNRNIDAVWIYTIISQDLSVVDDLIAKDTKGYFDIAIVYALKKYFSGQLEE
SSKELSKIKDKDNTRGIVPYLHLLIGRAMMPFSSQQAVHFFDYVRLTSPGTFLEEIALRNLLEI
TQNEVGERAFGYIRAYVTQFHHSIYKDHFISVLLRFFLHGQLKLPDEDIVFTISFFSLEEQRAI
YLKIAQNSVISGKRKIGFLAIKQLKRIIDRLDYKDLATIQLYENILNIPFVDIMSLQRSTCNIP
YYSLMEQDRYLKKASEIIMSEIGKSLIDIDFEHIQKDLLLDKKEPRHTNVSMGIESFIKKNRSQ
IESIDVLLAEAR
CLIBASIA_03120 protein from Candidatus Liberibacter asiaticus
(60 aa)
SEQ ID NO: 32
MARTQIALALSFFMITHSYYAFSQDEIKKNNPTLEKKPIVLMKHEIQEKKTLAAFTSFAS
CLIBASIA_04560 protein from Candidatus Liberibacter asiaticus
(195aa)
SEQ ID NO: 33
MKSKNILIVSTLVICVLSISSCDLGDSIAKKRNTIGNTIKKSINRVIQENNKPRNMTIFKTEVK
RDIRRASRLSLEEKSKNADKPTVIENQADNINIEVEVATNLNPNHQASEIDIAIENLPDLKSNH
QASEIDIAIENLPDLKSNHQASEIDIAIENLPDHQVDRNHTLSNLRGACYQPSLVSNSSLKLWD
VAF
CLIBASIA_05640 protein from Candidatus Liberibacter asiaticus
(68a)
SEQ ID NO: 34
MTIKKVLIASTLLSLCGCGLADEPKKLNPDQLCDAVCRLTLEEQKELQTKVNQRYEEHLTKGAK
LSSD
CLIBASIA_05320 protein from Candidatus Liberibacter asiaticus
(85aa)
SEQ ID NO: 35
MSKFVVRIMFLLSAISSNPILAANEHSSVSEQKRKETTVGFISRLVNKRPVANKRCPNATKQTP
PDHGSKYDTREVLMLFGGLNN

What is claimed is:

1. A method of detecting secreted protein in a citrus plant, the method comprising
forming a mixture of a sample from a citrus plant with a first antibody specific for a protein identical or substantially identical to a protein as listed in Table 1 or Table 2;
incubating the mixture with a polypeptide linked to solid support, wherein the polypeptide is the protein as listed in Table 1 or Table 2 or a protein comprising a fragment of at least 20 contiguous amino acids thereof;
washing unbound components of the mixture from the polypeptide linked to solid surface; and
detecting the presence or amount of first antibody bound the polypeptide linked to solid surface, thereby detecting secreted protein from Spiroplasma citri or Candidatus Liberibacter asiaticus.

2. The method of claim 1, wherein the first antibody is a polyclonal antibody, optionally generated by a method comprising immunizing a mammal with an adjuvant and the protein or an immunogenic fragment thereof and purifying antibody that specifically binds to the protein from blood of the mammal.

3. The method of claim 1, wherein the protein comprises SEQ ID NO:29 or a fragment of at least 20 contiguous amino acids thereof.

4. The method of claim 1, wherein the citrus plant is not artificially infected or graft-inoculated with a bacterial pathogen.

5. The method of claim 1, wherein the detecting comprises detecting the first antibody by contacting a labeled secondary antibody to the first antibody bound to the polypeptide linked to solid surface, washing away unbound secondary antibody, and determining an amount of secondary antibody specifically binding to the first antibody.

6. The method of claim 1, wherein the solid support is a well in a microtiter dish.

7. The method of claim 1, wherein the protein is detected and wherein the method further comprises destroying the plant.

8. A kit for detecting citrus stubborn disease or detecting citrus greening disease (Huanglongbing or HLB), the kit comprising a protein from Table 1 or Table 2 or a protein comprising a fragment of at least 20 contiguous amino acids thereof, wherein the protein is linked to a solid support.

9. The kit of claim 8, wherein the protein comprises SEQ ID NO:29 or a fragment of at least 20 contiguous amino acids thereof.

10. The kit of claim 8, further comprising a first antibody that specifically binds to the protein.

11. The kit of claim 8, wherein the first antibody is a polyclonal antibody, optionally generated by a method comprising immunizing a mammal with an adjuvant and the protein or an immunogenic fragment thereof and purifying antibody that specifically binds to the protein from blood of the mammal.

12. The kit of claim 8, further comprising a secondary antibody that binds to the first antibody.

13. The kit of claim 8, wherein the solid support is a well in a microtiter dish.
